SEQUENCE LISTING (72 sequences)

SEQ ID NO: 1 - protein, 14 aa, Homo sapiens
AVALLPAVLLALLA

SEQ ID NO: 2 - protein, 13 aa, synthetic construct
  VARIANT 8..9: note = X=leucine, isoleucine, or norleucine
  VARIANT 11..12: note = X=leucine, isoleucine, or norleucine
AAVAPAVXXAXXA

SEQ ID NO: 3 - protein, 15 aa, synthetic construct
AAVALLPAVLLALLA

SEQ ID NO: 4 - protein, 19 aa, synthetic construct
  VARIANT 1..2: note = X=lysine, arginine, or ornithine
  VARIANT 18..19: note = X=lysine, arginine, or ornithine
XXAAVALLPAVLLALLAXX

SEQ ID NO: 5 - protein, 19 aa, synthetic construct
KKAAVALLPAVLLALLAKK

SEQ ID NO: 6 - protein, 19 aa, synthetic construct
RRAAVALLPAVLLALLARR

SEQ ID NO: 7 - protein, 19 aa, synthetic construct
RRAAVALLPAVLLALLARK

SEQ ID NO: 8 - protein, 20 aa, synthetic construct
RKAAVALLPAVLLALLARKY

SEQ ID NO: 9 - protein, 28 aa, synthetic construct
AAVALLPAVLLALLAPCVQRKRQKLMPC

SEQ ID NO: 10 - protein, 21 aa, synthetic construct
  VARIANT 1..2: note = X=lysine, arginine, or ornithine
  VARIANT 5: note = X=valine, leucine, isoleucine, or norleucine
  VARIANT 7..8: note = X=valine, leucine, isoleucine, or norleucine
  VARIANT 11..13: note = X=valine, leucine, isoleucine, or norleucine
  VARIANT 15..16: note = X=valine, leucine, isoleucine, or norleucine
  REPEAT 18: note = repeat is 0, 1, 2, 3, or 4
  VARIANT 19..20: note = X=lysine, arginine, or ornithine
  VARIANT 21: note = X=tyrosine
  VARIANT 9: note = X=proline or arginine
  REPEAT 21: note = repeat is 0, 1, 2, 3, or 4
XXAAXAXXXAXXXAXXAPXXX

SEQ ID NO: 11 - protein, 21 aa, synthetic construct
  VARIANT 1..2: note = X=lysine, arginine, or ornithine
  VARIANT 19..20: note = X=lysine, arginine, or ornithine
  VARIANT 21: note = X=tyrosine
  REPEAT 21: note = repeat is 0, 1, 2, 3, or 4
XXAAVALLPAVLLALLAPXXX

SEQ ID NO: 12 - protein, 33 aa, synthetic construct
  VARIANT 1..2: note = X=lysine, arginine, or ornithine
  VARIANT 5: note = X=valine, leucine, isoleucine, or norleucine
  VARIANT 7..9: note = X=valine, leucine, isoleucine, or norleucine
  VARIANT 11..13: note = X=valine, leucine, isoleucine, or norleucine
  VARIANT 15..16: note = X=valine, leucine, isoleucine, or norleucine
  VARIANT 17..18: note = X=lysine, arginine, or ornithine
  VARIANT 19: note = X=tyrosine
  REPEAT 19: note = repeat is 0, 1, 2, 3, or 4
XXAAXAXXXAXXXAXXAPXXXCVQRKRQKLMPC

SEQ ID NO: 13 - protein, 26 aa, synthetic construct
AAVALLPAVLLALLAPVQRKRQKLMP

SEQ ID NO: 14 - protein, 28 aa, synthetic construct
  DISULFID 17..28: note = cyclized by disulfide bond at C17 and C28
AAVALLPAVLLALLAPCVQRKRQKLMPC

SEQ ID NO: 15 - protein, 14 aa, synthetic construct
  VARIANT 8..9: note = X=leucine, isoleucine, or norleucine
  VARIANT 11..12: note = X=leucine, isoleucine, or norleucine
AAVAPAVXXAXXAP

SEQ ID NO: 16 - protein, 16 aa, synthetic construct
AAVALLPAVLLALLAP

SEQ ID NO: 17 - protein, 10 aa, synthetic construct
  VARIANT 1: note = X=lysine
  VARIANT 2: note = X=arginine
  VARIANT 10: note = X=lysine
  REPEAT 1: note = repeat is 0, 1, 2, 3, or 4
  REPEAT 2: note = repeat is 0, 1, 2, 3, or 4
  REPEAT 10: note = repeat is 0, 1, 2, 3, or 4
XXAAVALLPX

SEQ ID NO: 18 - protein, 12 aa, synthetic construct
  VARIANT 1: note = X=lysine
  VARIANT 2: note = X=arginine
  VARIANT 12: note = X= lysine
  REPEAT 1: note = repeat is 0, 1, 2, 3, or 4
  REPEAT 2: note = repeat is 0, 1, 2, 3, or 4
  REPEAT 12: note = repeat is 0, 1, 2, 3, or 4
XXAVLLALLAPX

SEQ ID NO: 19 - protein, 19 aa, synthetic construct
  VARIANT 1: note = X= lysine
  VARIANT 2: note = X= arginine
  VARIANT 19: note = X= lysine
  REPEAT 1: note = repeat is 0, 1, 2, 3, or 4
  REPEAT 2: note = repeat is 0, 1, 2, 3, or 4
  REPEAT 19: note = repeat is 0, 1, 2, 3, or 4
XXAAVALLAAVLLALLAPX

SEQ ID NO: 20 - protein, 16 aa, synthetic construct
TTGTLLPGVLLALVVA

SEQ ID NO: 21 - protein, 19 aa, synthetic construct
  VARIANT 1: note = X=lysine
  VARIANT 2: note = X=arginine
  VARIANT 19: note = X=lysine
  REPEAT 1: note = repeat is 0, 1, 2, 3, or 4
  REPEAT 2: note = repeat is 0, 1, 2, 3, or 4
  REPEAT 19: note = repeat is 0, 1, 2, 3, or 4
XXTTGTLLPGVLLALVVAX

SEQ ID NO: 22 - protein, 16 aa, synthetic construct
TTGTLLPRVLLALVVA

SEQ ID NO: 23 - protein, 19 aa, synthetic construct
  VARIANT 1: note = X=lysine
  VARIANT 2: note = X=arginine
  VARIANT 19: note = X=lysine
  REPEAT 1: note = repeat is 0, 1, 2, 3, or 4
  REPEAT 2: note = repeat is 0, 1, 2, 3, or 4
  REPEAT 19: note = repeat is 0, 1, 2, 3, or 4
XXTTGTLLPGVLLALVVAX

SEQ ID NO: 24 - protein, 10 aa, synthetic construct
  VARIANT 1: note = X=lysine
  VARIANT 2: note = X=arginine
  VARIANT 10: note = X=lysine
  REPEAT 1: note = repeat is 0, 1, 2, 3, or 4
  REPEAT 2: note = repeat is 0, 1, 2, 3, or 4
  REPEAT 10: note = repeat is 0, 1, 2, 3, or 4
XXTTGTLLPX

SEQ ID NO: 25 - protein, 11 aa, synthetic construct
  VARIANT 1: note = X=lysine
  VARIANT 2: note = X=arginine
  VARIANT 11: note = X=lysine
  REPEAT 1: note = repeat is 0, 1, 2, 3, or 4
  REPEAT 2: note = repeat is 0, 1, 2, 3, or 4
  REPEAT 11: note = repeat is 0, 1, 2, 3, or 4
XXVLLALVVAX

SEQ ID NO: 26 - protein, 19 aa, synthetic construct
  VARIANT 19: note = X=lysine
  REPEAT 19: note = repeat is 0, 1, 2, 3, or 4
RAAVALLPAVLLALLAPYX

SEQ ID NO: 27 - protein, 19 aa, synthetic construct
  VARIANT 19: note = X= lysine
  REPEAT 19: note = repeat is 0, 1, 2, 3, or 4
RAAVALLPAVLLAVLAPYX

SEQ ID NO: 28 - protein, 32 aa, synthetic construct
  VARIANT 1: note = X=lysine
  VARIANT 2: note = X=arginine
  VARIANT 31: note = X=lysine
  REPEAT 32: note = repeat is 0, 1, 2, 3, or 4
  REPEAT 1: note = repeat is 0, 1, 2, 3, or 4
  REPEAT 2: note = repeat is 0, 1, 2, 3, or 4
  REPEAT 31: note = repeat is 0, 1, 2, 3, or 4
XXAAVALLPAVLLALLAPDVRKRQDLEQKMXY

SEQ ID NO: 29 - protein, 19 aa, synthetic construct
  VARIANT 1: note = X= lysine
  VARIANT 2: note = X= arginine
  VARIANT 19: note = X= lysine
  REPEAT 1: note = repeat is 0, 1, 2, 3, or 4
  REPEAT 2: note = repeat is 0, 1, 2, 3, or 4
  REPEAT 19: note = repeat is 0, 1, 2, 3, or 4
XXAAVALLPAVLLALLAPX

SEQ ID NO: 30 - protein, 19 aa, synthetic construct
  VARIANT 1: note = X=lysine
  VARIANT 2: note = X=arginine
  VARIANT 19: note = X=lysine
  REPEAT 1: note = repeat is 0, 1, 2, 3, or 4
  REPEAT 2: note = repeat is 0, 1, 2, 3, or 4
  REPEAT 19: note = repeat is 0, 1, 2, 3, or 4
XXAAVALLAAVLLALLAPX

SEQ ID NO: 31 - protein, 19 aa, synthetic construct
  VARIANT 1: note = X=lysine
  VARIANT 2: note = X=arginine
  VARIANT 19: note = X=lysine
  REPEAT 1: note = repeat is 0, 1, 2, 3, or 4
  REPEAT 2: note = repeat is 0, 1, 2, 3, or 4
  REPEAT 19: note = repeat is 0, 1, 2, 3, or 4
XXTTGTLLPGVLLALVVAX

SEQ ID NO: 32 - protein, 19 aa, synthetic construct
  VARIANT 1: note = X=lysine
  VARIANT 2: note = X=arginine
  VARIANT 19: note = X=lysine
  REPEAT 1: note = repeat is 0, 1, 2, 3, or 4
  REPEAT 2: note = repeat is 0, 1, 2, 3, or 4
  REPEAT 19: note = repeat is 0, 1, 2, 3, or 4
XXTTGTLLPRVLLALVVAX

SEQ ID NO: 33 - protein, 10 aa, synthetic construct
  VARIANT 1: note = X=lysine
  VARIANT 2: note = X=arginine
  VARIANT 10: note = X=lysine
  REPEAT 1: note = repeat is 0, 1, 2, 3, or 4
  REPEAT 2: note = repeat is 0, 1, 2, 3, or 4
  REPEAT 10: note = repeat is 0, 1, 2, 3, or 4
XXTTGTLLPX

SEQ ID NO: 34 - protein, 12 aa, synthetic construct
  VARIANT 1: note = X=lysine
  VARIANT 2: note = X=arginine
  VARIANT 12: note = X=lysine
  REPEAT 1: note = repeat is 0, 1, 2, 3, or 4
  REPEAT 2: note = repeat is 0, 1, 2, 3, or 4
  REPEAT 12: note = repeat is 0, 1, 2, 3, or 4
XXGVLLALVVAX

SEQ ID NO: 35 - protein, 29 aa, synthetic construct
  VARIANT 1: note = X=lysine
  VARIANT 2: note = X=arginine
  REPEAT 1: note = repeat is 0, 1, 2, 3, or 4
  REPEAT 2: note = repeat is 0, 1, 2, 3, or 4
XXAAVALLPAVLLALLAPCVQKRQKLMPC

SEQ ID NO: 36 - protein, 20 aa, synthetic construct
KKAAVALLPAVLLALLAPKK

SEQ ID NO: 37 - protein, 20 aa, synthetic construct
RRAAVALLPAVLLALLAPRR

SEQ ID NO: 38 - protein, 20 aa, synthetic construct
RRAAVALLPAVLLALLAPRK

SEQ ID NO: 39 - protein, 21 aa, synthetic construct
RKAAVALLPAVLLALLAPRKY

SEQ ID NO: 40 - protein, 11 aa, synthetic construct
KRAAVALLPKK

SEQ ID NO: 41 - protein, 13 aa, synthetic construct
KRAVLLALLAPKK

SEQ ID NO: 42 - protein, 20 aa, synthetic construct
KRAAVALLAAVLLALLAPKK

SEQ ID NO: 43 - protein, 20 aa, synthetic construct
KRTTGTLLPGVLLALVVAKK

SEQ ID NO: 44 - protein, 20 aa, synthetic construct
KRTTGTLLPGVLLALVVAKK

SEQ ID NO: 45 - protein, 11 aa, synthetic construct
KRTTGTLLPKK

SEQ ID NO: 46 - protein, 12 aa, synthetic construct
KRVLLALVVAKK

SEQ ID NO: 47 - protein, 11 aa, synthetic construct
KRAAVALLPKK

SEQ ID NO: 48 - protein, 16 aa, synthetic construct
AAVALLPAVLLALLAP

SEQ ID NO: 49 - protein, 29 aa, synthetic construct
AAVALLPAVLLALLAPCYVQRKRQKLMPC

SEQ ID NO: 50 - protein, 28 aa, synthetic construct
AAVALLPAVLLAVLAPCVQRKRQKLMPC

SEQ ID NO: 51 - protein, 28 aa, synthetic construct
AAVALLPAVLLALLAPCVQRDEQKLMPC

SEQ ID NO: 52 - protein, 28 aa, synthetic construct
AAVALLPAVLLAVLAPCVQRDEQKLMPC

SEQ ID NO: 53 - protein, 21 aa, synthetic construct
  VARIANT 1..2: note = X=lysine, arginine, or ornithine
  VARIANT 5: note = X=valine, leucine, isoleucine, or norleucine
  VARIANT 7..8: note = X=valine, leucine, isoleucine, or norleucine
  VARIANT 11..13: note = X=valine, leucine, isoleucine, or norleucine
  VARIANT 15..16: note = X=valine, leucine, isoleucine, or norleucine
  REPEAT 18: note = Repeat is 0, 1, 2, 3, or 4
  VARIANT 19..20: note = X=lysine, arginine, or ornithine
  VARIANT 21: note = X=tyrosine
  REPEAT 21: note = repeat is 0, 1, 2, 3, or 4
XXAAXAXXPAXXXAXXAPXXX

SEQ ID NO: 54 - protein, 17 aa, synthetic construct
LLAAVALLPAVLLALLA

SEQ ID NO: 55 - protein, 17 aa, synthetic construct
AAVALLPAVLLALLALL

SEQ ID NO: 56 - protein, 19 aa, synthetic construct
LLAAVALLPAVLLALLALL

SEQ ID NO: 57 - protein, 18 aa, synthetic construct
LLAAVALLPAVLLALLAP

SEQ ID NO: 58 - protein, 18 aa, synthetic construct
AAVALLPAVLLALLAPLL

SEQ ID NO: 59 - protein, 20 aa, synthetic construct
LLAAVALLPAVLLALLAPLL

SEQ ID NO: 60 - protein, 17 aa, synthetic construct
  VARIANT 1: note = X=Lysine
  VARIANT 17: note = X=Lysine
  REPEAT 1: note = repeat is 0, 1, 2, 3, or 4
  REPEAT 17: note = repeat is 0, 1, 2, 3, or 4
XAAVALLPAVLLALLAX

SEQ ID NO: 61 - protein, 18 aa, synthetic construct
  VARIANT 1: note = X=Lysine
  VARIANT 18: note = X=Lysine
  REPEAT 1: note = repeat is 0, 1, 2, 3, or 4
  REPEAT 18: note = repeat is 0, 1, 2, 3, or 4
XAAVALLPAVLLALLAPX

SEQ ID NO: 62 - protein, 32 aa, synthetic construct
  REPEAT 1: note = repeat is 0, 1, 2, 3, or 4
  REPEAT 17: note = repeat is 0, 1, 2, 3, or 4
  VARIANT 1: note = X=Lysine
  VARIANT 17: note = X=Lysine
XAAVALLPAVLLALLAXDVRKRQDLEQKMKKY

SEQ ID NO: 63 - protein, 33 aa, synthetic construct
  REPEAT 1: note = repeat is 0, 1, 2, 3, or 4
  REPEAT 18: note = repeat is 0, 1, 2, 3, or 4
  VARIANT 1: note = X=Lysine
  VARIANT 18: note = X=Lysine
XAAVALLPAVLLALLAPXDVRKRQDLEQKMKKY

SEQ ID NO: 64 - protein, 27 aa, synthetic construct
  REPEAT 1: note = repeat is 0, 1, 2, 3, or 4
  REPEAT 17: note = repeat is 0, 1, 2, 3, or 4
  VARIANT 1: note = X=Lysine
  VARIANT 17: note = X=Lysine
XAAVALLPAVLLALLAXKTGLRRLQHR

SEQ ID NO: 65 - protein, 28 aa, synthetic construct
  REPEAT 1: note = repeat is 0, 1, 2, 3, or 4
  REPEAT 18: note = repeat is 0, 1, 2, 3, or 4
  VARIANT 1: note = X=lysine
  VARIANT 18: note = X=lysine
XAAVALLPAVLLALLAPXKTGLRRLQHR

SEQ ID NO: 66 - protein, 30 aa, synthetic construct
AAVALLPAVLLALLADVRKRQDLEQKMKKY

SEQ ID NO: 67 - protein, 31 aa, synthetic construct
AAVALLPAVLLALLAPDVRKRQDLEQKMKKY

SEQ ID NO: 68 - protein, 25 aa, synthetic construct
AAVALLPAVLLALLAKTGLRRLQHR

SEQ ID NO: 69 - protein, 26 aa, synthetic construct
AAVALLPAVLLALLAPKTGLRRLQHR

SEQ ID NO: 70 - protein, 12 aa, synthetic construct
CVQRKRQKLMPC

SEQ ID NO: 71 - protein, 33 aa, synthetic construct
  VARIANT 1: note = X=Lys, Arg, or Orn
  VARIANT 2: note = X=Lys, Arg, or Orn
  VARIANT 19: note = X=Lys, Arg, or Orn
  VARIANT 20: note = X=Lys, Arg, or Orn
  VARIANT 5: note = X=Val, Leu, Ile, or Nle
  VARIANT 7: note = X=Val, Leu, Ile, or Nle
  VARIANT 8: note = X=Val, Leu, Ile, or Nle
  VARIANT 11: note = X=Val, Leu, Ile, or Nle
  VARIANT 12: note = X=Val, Leu, Ile, or Nle
  VARIANT 13: note = X=Val, Leu, Ile, or Nle
  REPEAT 15: note = repeat is 0, 1, 2, 3, or 4
  VARIANT 21: note = X=Tyr
  REPEAT 21: note = repeat is 0, 1, 2, 3, or 4
XXAAXAXXPAXXXAXXAPXXXCVQRKRQKLMPC

SEQ ID NO: 72 - protein, 27 aa, synthetic construct
AAVALLPAVLLALLACVQRKRQKLMPC

(19) United States
(12) Patent Application Publication
(10) Pub. No.: US 2025/0295793 A1
(43) Pub. Date: Sep. 25, 2025

(54) PEPTIDE-DRUG CONJUGATES AND USES THEREOF

(71) Applicant: Amytrx Therapeutics, Inc., Birmingham, AL (US)

(72) Inventors: Vernon L. Alvarez, Nashville, TN (US); Matthew A. Gonda, Birmingham, AL (US)

(21) Appl. No.: 19/105,995

(22) PCT Filed: Aug. 23, 2023
(86) PCT No.: PCT/US2023/072733

§ 371 Date: Feb. 24, 2025

Related U.S. Application Data
(60) Provisional application No. 63/400,373, filed on Aug. 23, 2022.

(51) Int. Cl. A61K 47/64; A61K 31/519; C07K 14/00

(52) U.S. Cl. CPC A61K 47/64; A61K 31/519; C07K 14/001

(57) ABSTRACT

Conjugates comprising leukocyte-targeting molecules chemically conjugated to one or more active pharmaceutical agents are provided herein, as well as uses thereof.

### RELATED APPLICATIONS

This application claims the benefit of U.S. Provisional patent application 63/400,373 filed Aug. 23, 2022, the entire contents of which are incorporated by reference herein.

### SEQUENCE LISTING

This application contains a sequence listing having the filename 1959919-00008_Sequence_Listing.xml, which is 127,534 bytes in size, and was created on Aug. 22, 2023. The entire content of this sequence listing is incorporated herein by reference.

### SUMMARY

Provided herein are conjugates comprising leukocyte-targeting molecules chemically conjugated to one or more active pharmaceutical agent. The conjugates provided herein are useful in delivering the one or more active pharmaceutical agent to leukocytes in a subject in need thereof or for treating a disease in the subject that the one or more active pharmaceutical agents are useful in treating. The conjugates are also useful in treating leukocyte associated diseases, cancers, immune diseases, autoimmune diseases, inflammatory diseases, or neurodegenerative diseases.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1 depicts an exemplary conjugation of active pharmaceutical agent methotrexate to SEQ ID NO:9 or SEQ ID NO: 14 (cyclized).

FIG. 2 shows an example of a peptide coupling to methotrexate.

### DETAILED DESCRIPTION

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, all numerical values provided herein, unless otherwise indicated, are to be understood as being modified by the term “about.” Accordingly, the last decimal place of a numerical value provided herein indicates its degree of accuracy. Where no other error margins are given, the maximum margin is ascertained by applying the rounding-off convention to the last decimal place or last significant digit when a decimal is not present in the given numerical value.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The terms “composition” and “pharmaceutical composition” refer to a mixture of at least one compound described herein with a pharmaceutically acceptable carrier. The pharmaceutical composition facilitates administration of the compound to a patient or subject. Multiple techniques of administering a compound exist including, but not limited to, intravenous, oral, aerosol, parenteral, ophthalmic, nasal, pulmonary, and topical administration.

The terms “effective amount” and “therapeutically effective amount” refer to an amount of active ingredient, such as a compound described herein, administered to a subject, either as a single dose or as part of a series of doses, which produces a desired effect. In general, the effective amount can be estimated initially either in cell culture assays or in mammalian animal models, for example, in non-human primates, murines (for example, mice), leporines (for example, rabbits), canines (for example, dogs), bovines, assinines, equines, cervines or elaphines or rusines, felines, ursines, ovines (for example, sheep), hircines (for example, goats), or pigs. The animal model may also be used to determine the appropriate concentration range and route of administration. Such information can then be used to determine useful doses and routes for administration in non-human subjects and human subjects.

The term “pharmaceutically acceptable carrier” means a pharmaceutically acceptable material, composition or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical formulations described herein are known in the art and described, for example, in “Remington's Pharmaceutical Sciences” (Genaro (Ed.), Mack Publishing Co., 1985), the entire content of which is incorporated herein by reference.

The term “pharmaceutically acceptable salt” refers to derivatives of the disclosed compounds wherein the parent compound is modified by converting an existing acid or base moiety to its salt form. Pharmaceutically acceptable salts can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two solvents. Lists of suitable salts are found in “Handbook of Pharmaceutical Salts: Properties, Selection, and Use” (P. Henrich Stahl & Camille G. Wermuth (Eds.), VHCA & Wiley-VCH, 2002), the entire content of which is incorporated herein by reference.

The term “plasma” as used herein refers to whole blood depleted of red blood cells.

The term “refractory disease” refers to a disease that continues to progress during treatment with a pharmaceutical ingredient other than the compounds provided herein, partially responds to the other treatment, or transiently responds to the other treatment. The term may be applied to each of the diseases referred to herein.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the described subject matter and does not pose a limitation on the scope of the subject matter otherwise claimed. No language in the specification should be construed as indicating any non-claimed element essential to practicing the described subject matter.

Groupings of alternative elements or embodiments of this disclosure are not to be construed as limitations. Each group member may be referred to and claimed individually or in any combination with other members of the group or other elements found herein. Furthermore, a recited member of a group may be included in, or excluded from, another recited group for reasons of convenience or patentability.

Reference made to a patent document or other publication in this specification serves as an incorporation herein by reference of the entire content of such document or publication.

Embodiments of this disclosure are illustrative. Accordingly, the present disclosure is not limited to that precisely as shown and described.

### Conjugates

Provided herein are covalently linked conjugates of a leukocyte-targeting molecule to one or more active pharmaceutical agents, optionally wherein the covalent linkage is by a direct bond or via a linker moiety. International Application No. PCT/US2022/075348 (WO2023028486)—the entire content of which is incorporated herein by reference—describes features, methods, and uses of leukocyte-targeting molecules herein.

In some embodiments, the conjugates provided herein include the formula:

Z—(X-J)ₙ
or a pharmaceutically acceptable salt thereof,
wherein:
Z is a leukocyte-targeting molecule;
X is, independently, a direct bond or a linker;
J is, independently, an active pharmaceutical agent; and
n is 1, 2, 3, or 4, or more than 4.

In some embodiments, the conjugates provided herein include the formula:

Z—X-J
or a pharmaceutically acceptable salt thereof,
wherein:
Z is a leukocyte-targeting molecule;
X is a direct bond or a linker; and
J is an active pharmaceutical agent.

In some embodiments, the leukocyte-targeting molecule comprises an amino acid sequence selected from Table 1.

TABLE 1
Leukocyte-targeting molecule sequences.

| SEQ ID NO: | SEQUENCE |
|---|---|
| 1 | AVALLPAVLLALLA |
| 2 | AAVAPAVX¹⁶X¹⁶AX¹⁶X¹⁶A |
| 3 | AAVALLPAVLLALLA |
| 4 | X¹X¹AAVALLPAVLLALLAX¹X¹ |
| 5 | KKAAVALLPAVLLALLAKK |
| 6 | RRAAVALLPAVLLALLARR |
| 7 | RRAAVALLPAVLLALLARK |
| 8 | RKAAVALLPAVLLALLARKY |
| 9 | AAVALLPAVLLALLAPCVQRKRQKLMPC |
| 10 | X¹X²AAX³AX⁴X⁵X¹⁷AX⁶X7X⁸AX⁹X¹⁰A(P)ₙX¹¹X¹²(X¹³)ₙ |
| 11 | X²X²AAVALLPAVLLALLAPX¹¹X¹²(X¹³)ₙ |
| 12 | X¹X²AAX³AX⁴X⁵X¹⁷AX⁶X7X⁸AX⁹X¹⁰APX¹¹X¹²(X¹³)ₙCVQRKRQKLMPC |
| 13 | AAVALLPAVLLALLAPVQRKRQKLMP |
| 14 | AAVALLPAVLLALLAPCVQRKRQKLMPC (cyclized by SS bond at C¹⁷ and C²⁸) |
| 15 | AAVAPAVX¹⁶X¹⁶AX¹⁶X¹⁶AP |
| 16 | AAVALLPAVLLALLAP |
| 17 | (X¹⁴)ₙ(X¹⁵)ₙAAVALLP(X¹⁴)ₘ |
| 18 | (X¹⁴)ₙ(X¹⁵)ₙAVLLALLAP(X¹⁴)ₘ |
| 19 | (X¹⁴)ₙ(X¹⁵)ₙAAVALLAAVLLALLAP(X¹⁴)ₘ |
| 20 | TTGTLLPGVLLALVVA |
| 21 | (X¹⁴)ₙ(X¹⁵)ₙTTGTLLPGVLLALVVA(X¹⁴)ₘ |
| 22 | TTGTLLPRVLLALVVA |
| 23 | (X¹⁴)ₙ(X¹⁵)ₙTTGTLLPGVLLALVVA(X¹⁴)ₘ |
| 24 | (X¹⁴)ₙ(X¹⁵)ₙTTGTLLP(X¹⁴)ₘ |
| 25 | (X¹⁴)ₙ(X¹⁵)ₙVLLALVVA(X¹⁴)ₘ |
| 26 | RAAVALLPAVLLALLAPY(X¹⁴)ₘ |
| 27 | RAAVALLPAVLLAVLAPY(X¹⁴)ₘ |
| 28 | (X¹⁴)ₙ(X¹⁵)ₙAAVALLPAVLLALLAPDVRKRQDLEQ KM(X¹⁴)_{z}(Y)ₘ |
| 29 | (X¹⁴)ₙ(X¹⁵)ₙAAVALLPAVLLALLAP(X¹⁴)ₘ |
| 30 | (X¹⁴)ₙ(X¹⁵)ₙAAVALLAAVLLALLAP(X¹⁴)ₘ |
| 31 | (X¹⁴)ₙ(X¹⁵)ₙTTGTLLPGVLLALVVA(X¹⁴)ₘ |
| 32 | (X¹⁴)ₙ(X¹⁵)ₙTTGTLLPRVLLALVVA(X¹⁴)ₘ |
| 33 | (X¹⁴)ₙ(X¹⁵)ₙTTGTLLP(X¹⁴)ₘ |
| 34 | (X¹⁴)ₙ(X¹⁵)ₙGVLLALVVA(X¹⁴)ₘ |
| 35 | (X¹⁴)ₙ(X¹⁵)ₙAAVALLPAVLLALLAPCVQKRQKLMPC |
| 36 | KKAAVALLPAVLLALLAPKK |
| 37 | RRAAVALLPAVLLALLAPRR |
| 38 | RRAAVALLPAVLLALLAPRK |
| 39 | RKAAVALLPAVLLALLAPRKY |
| 40 | KRAAVALLPKK |
| 41 | KRAVLLALLAPKK |
| 42 | KRAAVALLAAVLLALLAPKK |
| 43 | KRTTGTLLPGVLLALVVAKK |
| 44 | KRTTGTLLPGVLLALVVAKK |
| 45 | KRTTGTLLPKK |
| 46 | KRVLLALVVAKK |
| 47 | KRAAVALLPKK |
| 48 | AAVALLPAVLLALLAP |
| 49 | AAVALLPAVLLALLAPCYVQRKRQKLMPC |
| 50 | AAVALLPAVLLAVLAPCVQRKRQKLMPC |
| 51 | AAVALLPAVLLALLAPCVQRDEQKLMPC |
| 52 | AAVALLPAVLLAVLAPCVQRDEQKLMPC |
| 53 | X¹X²AAX³AX⁴X⁵PAX⁶X⁷X⁸AX⁹X¹⁰A(P)ₘX¹¹X¹²(X¹³)ₙ |
| 54 | LLAAVALLPAVLLALLA |
| 55 | AAVALLPAVLLALLALL |
| 56 | LLAAVALLPAVLLALLALL |
| 57 | LLAAVALLPAVLLALLAP |
| 58 | AAVALLPAVLLALLAPLL |
| 59 | LLAAVALLPAVLLALLAPLL |
| 60 | (X¹⁴)ₙAAVALLPAVLLALLA(X¹⁴)ₙ |
| 61 | (X¹⁴)ₙAAVALLPAVLLALLAP(X¹⁴)ₙ |
| 62 | (X¹⁴)ₙAAVALLPAVLLALLA(X¹⁴)ₙDVRKRQDLEQKMKKY |
| 63 | (X¹⁴)ₙAAVALLPAVLLALLAP(X¹⁴)ₙDVRKRQDLEQKMKKY |
| 64 | (X¹⁴)ₙAAVALLPAVLLALLA(X¹⁴)ₙKTGLRRLQHR |
| 65 | (X¹⁴)ₙAAVALLPAVLLALLAP(X¹⁴)ₙKTGLRRLQHR |
| 66 | AAVALLPAVLLALLADVRKRQDLEQKMKKY |
| 67 | AAVALLPAVLLALLAPDVRKRADLEQKMKKY |
| 68 | AAVALLPAVLLALLAKTGLRRLQHR |
| 69 | AAVALLPAVLLALLAPKTGLRRLQHR |
| 70 | CVQRKRQKLMPC |
| 71 | X¹X²AAX³AX⁴X⁵PAX⁶X⁷X⁸AX⁹X¹⁰A(P)ₘX¹¹X¹²(X¹³)ₙCVQRKRQKLMPC |
| 72 | AAVALLPAVLLALLACVQRKRQKLMPC |

Each X¹, X², X¹¹, and X¹² is, independently, Lys, Arg, or Orn
Each X³, X⁴, X⁵, X⁶, X⁷, X⁸, X⁹, X¹⁰ is, independently, Val, Leu, Ile, or Nle
Each X¹³ is, independently, Tyr
Each X¹⁴ is, independently, Lys
Each X¹⁵ is, independently, Arg
Each X¹⁶ is, independently, Leu, Ile, or Nle
Each X¹⁷ is, independently, Pro or Ala
Each ⁿ is, independently, 0, 1, 2, 3, or 4
Each ^{m} is, independently, 0, 1, 2, 3, or 4

In some embodiments, the peptide sequences of Table 1 may be linear or cyclized by a disulfide bond between two cysteine residues.

In some embodiments, the leukocyte-targeting molecule is an amino acid sequence of a formula selected from SEQ ID NO:2-SEQ ID NO:72, optionally including a disulfide bond between the thiols of two cysteine amino acids of the leukocyte-targeting molecule.

In some embodiments, the leukocyte-targeting molecule comprises an amino acid sequence of the formula:

X¹X²AAX³AX⁴X⁵PAX⁶X⁷X⁸AX⁹X¹⁰A(P)ₘX¹¹X¹²(X¹³)ₙ (SEQ ID NO:53)
or a pharmaceutically acceptable salt thereof.

In some embodiments, the leukocyte-targeting molecule is

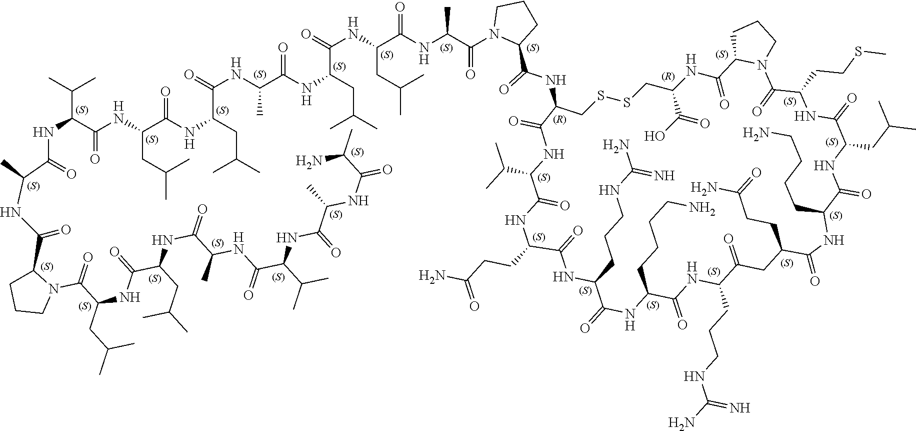

or a pharmaceutically acceptable salt thereof. In some embodiments, the leukocyte-targeting molecule is in a salt form. In some embodiments, the salt form of the leukocyte-targeting molecule includes at least one acetate counterion. In some embodiments of the conjugates provided herein, the leukocyte-targeting molecule is conjugated to one, two, three, or four moieties comprising an active pharmaceutical agent. In some embodiments, the leukocyte-targeting molecule is conjugated to one moiety comprising an active pharmaceutical agent, e.g., in some embodiments n is 1. In some embodiments, each moiety is, independently, covalently conjugated to an —SH, —COOH, or —NH₂ group of the leukocyte-targeting molecule, for example, at an amino-acid side chain of the leukocyte-targeting molecule, for example, the amine on the side chain of a lysine or the —SH on the side chain of a cysteine. In some embodiments, X-J is connected to Z by an amide or disulfide covalent connection. In some embodiments, X is a direct bond, and J is connected to Z by a disulfide or amide covalent connection. In some embodiments, Z is an amino-acid sequence shown in Table 1.

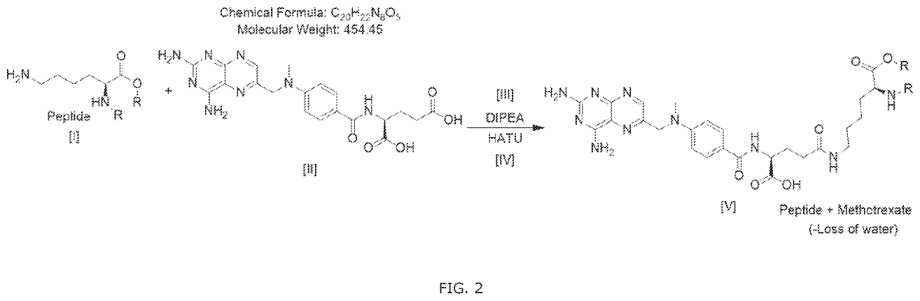

In some embodiments, Peptide (I) of FIG. 2 is a lysine-containing leukocyte-targeting molecule provided herein, including those of Table 1.

In some embodiments of the conjugates provided herein, an X-J comprises a nuclease, e.g., a ribonuclease or a deoxyribonuclease. In some embodiments, an X-J comprises an active pharmaceutical agent having a molecular weight of less than 1,000 kD, for example, less than 600 kD, for example less than 400 kD, and optionally, a molecular weight of at least 100 kD, and optionally including a —COOH moiety.

In some embodiments of the conjugates provided herein, the active pharmaceutical agent comprises a methotrexate. In some embodiments of the conjugates provided herein, J or X-J comprises a formula:

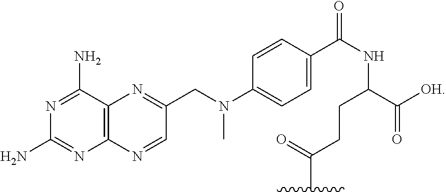 [SMILES: *C(=O)CCC(NC(=O)c1ccc(N(C)Cc2cnc3nc(N)nc(N)c3n2)cc1)C(=O)O]

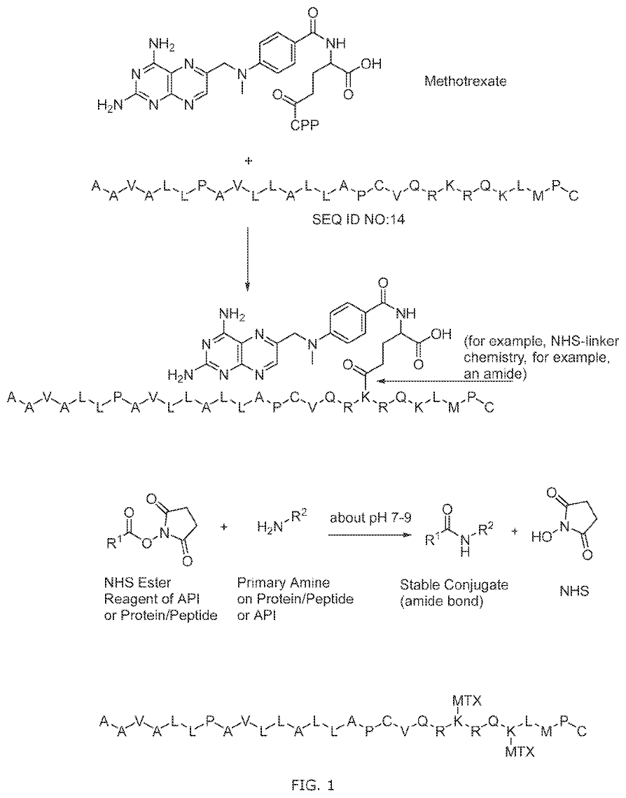

Similarly, an active pharmaceutical agent comprising a —COOH group, such as is found on methotrexate, can be conjugated to lysine-containing leukocyte-targeting molecules, including those in Table 1, provided herein. Such an active pharmaceutical agent may, in some embodiments, be conjugated to a leukocyte-targeting molecule as described herein, for example as shown in FIG. 1 or FIG. 2.

In some embodiments of the conjugates provided herein, Z comprises at least one lysine or at least one cysteine residue, and the side chain of the lysine is connected to X-J by an amide covalent connection or the side chain of the cysteine is connected to X-J by a disulfide covalent connection.

In some embodiments of the conjugates provided herein, each X-J, independently, comprises an RNase,

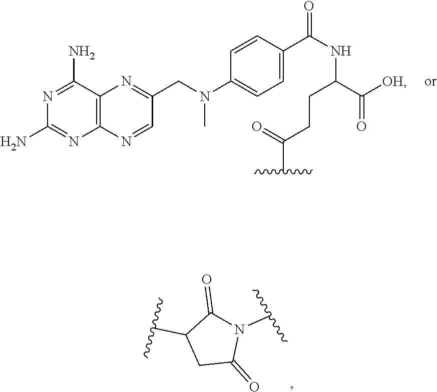 [SMILES: *C(=O)CCC(NC(=O)c1ccc(N(C)Cc2cnc3nc(N)nc(N)c3n2)cc1)C(=O)O ; *C1CC(=O)N(*)C1=O]

or a combination thereof.

In some embodiments regarding the leukocyte-targeting molecule, X³ and X⁶ are valine. In some embodiments, X¹, X⁵, X⁷, X⁸, X⁹, and X¹⁰ are each, independently, leucine, isoleucine, or norleucine. In some embodiments, the leukocyte-targeting molecule comprises an amino acid sequence of the formula: X¹X²AAVALLPAVLLALLA(P)ₘX²¹X¹²(X¹³)ₙ (SEQ ID NO: 11), or a pharmaceutically acceptable salt thereof. In some embodiments, the leukocyte-targeting molecule comprises at least one ornithine, isoleucine, or norleucine. In some embodiments, the leukocyte-targeting molecule comprises an amino acid sequence selected from KKAAVALLPAVLLALLAKK (SEQ ID NO:5), RRAAVALLPAVLLALLARR (SEQ ID NO:6), RRAAVALLPAVLLALLARK (SEQ ID NO:7), RKAAVALLPAVLLALLARKY (SEQ ID NO:8), KKAAVALLPAVLLALLAPKK (SEQ ID NO:36), RRAAVALLPAVLLALLAPRR (SEQ ID NO:37), RRAAVALLPAVLLALLAPRK (SEQ ID NO:38), or RKAAVALLPAVLLALLAPRKY (SEQ ID NO:39). In some embodiments, the leukocyte-targeting molecule further comprises the amino acid sequence CVQRKRQKLMPC (SEQ ID NO:70). In some embodiments, the leukocyte-targeting molecule comprises an amino acid sequence of the formula: X¹X²AAX³AX⁴X⁵PAX⁶X⁷X⁸AX⁹X¹⁰A(P)ₘX¹¹X¹²(X¹³)ₙCVQRKRQKLMPC (SEQ ID NO:71), or a pharmaceutically acceptable salt thereof. In some embodiments, the leukocyte-targeting molecule comprises at least one iodine. In some embodiments, the at least one iodine is selected, independently, from ¹²³I, ¹²⁴I, ¹²⁵I, or ¹³¹I. In some embodiments, the leukocyte-targeting molecule comprises at least one D-amino acid. In some embodiments, the leukocyte-targeting molecule comprises an amino acid sequence AAVALLPAVLLALLACVQRKROKLMPC (SEQ ID NO:72). In some embodiments, the leukocyte-targeting molecule comprises an amino acid sequence AAVALLPAVLLALLAPCVQRKRQKLMPC (SEQ ID NO:9). In some embodiments, the leukocyte-targeting molecule comprises a cyclic peptide. In some embodiments, the leukocyte-targeting molecule comprises a cysteine at position 16 and 17 of the amino acid sequence, or at position 17 and 28 of the amino acid sequence, and C¹⁶ and C²⁷ or C¹⁷ and C²⁸ are covalently linked. In some embodiments, the leukocyte-targeting molecule comprises two cysteine amino acids that are covalently linked by a disulfide bond.

In some embodiments, the leukocyte-targeting molecule is covalently linked to an active pharmaceutical agent by a direct bond. In some embodiments, the leukocyte-targeting molecule is covalently linked to an active pharmaceutical agent by a linker moiety.

Covalent linkage of an active pharmaceutical agent to the leukocyte-targeting molecule may, in some embodiments, be performed using linker chemistry, for example, N-hydroxysuccinamide (NHS) chemistry. Thus, in some embodiments, an active pharmaceutical agent includes a carboxylic acid that is activated with succinamide, the activated moiety is then coupled to an amine on the compound, for example, the N-terminus or the side chain amine of a lysine, glycine, asparagine, or glutamine, in order to form an amide bond between the active pharmaceutical agent and the leukocyte-targeting molecule. In some embodiments, the active pharmaceutical agent comprises an amine and the leukocyte-targeting molecule comprises a carboxylic acid, whereby NHS chemistry is similarly used to couple the two together. Thus, in some embodiments, the active pharmaceutical agent comprises an amine (for example, a primary amine) and the leukocyte-targeting molecule comprises a carboxylic acid, for example the C-terminus or the side chain carboxylic acid of an aspartic acid or glutamic acid. In some embodiments, the active pharmaceutical agent comprises a carboxylic acid and the leukocyte-targeting molecule comprises an amine (for example, a primary amine). The carboxylic acid and the amine may be covalently linked via a linker chemistry, for example, NHS chemistry, to form a covalent linkage, for example, an amide linkage. One or more active pharmaceutical agents may be coupled to the leukocyte-targeting molecule. Thus, in some embodiments, one, two, or three, or more active pharmaceutical agents may be coupled to a leukocyte-targeting molecule. In some embodiments, the number of active pharmaceutical agents coupled to a leukocyte-targeting molecule corresponds to the number of primary amine residues, carboxylic acid residues, cysteine residues, or a combination thereof, in the leukocyte-targeting molecule. A single leukocyte-targeting molecule may include one, two, or three, or more types of chemically different active pharmaceutical agents. For example, a first active pharmaceutical agent may be coupled to the leukocyte-targeting molecule, and then a second active pharmaceutical agent, different in structure than the first active pharmaceutical agent, may be coupled to the leukocyte-targeting molecule. Further active pharmaceutical agents, for example, a third, a fourth, and so on, active pharmaceutical agent may, in some embodiments, be serially coupled to the leukocyte-targeting molecule. Alternatively, in some embodiments, one or more active pharmaceutical agents may be coupled in one pot to a leukocyte-targeting molecule. FIG. 1 provides one example of an active pharmaceutical agent coupled to a leukocyte-targeting molecule. Thus, in some embodiments, provided herein is a compound comprising SEQ ID NO:9 or SEQ ID NO: 14 coupled to one or two methotrexates.

Exemplary linkers for covalent linkage include cleavable linkers and non-cleavable linkers. In some embodiments, a linker can assist in the delivery and accurate release of pharmaceutical agents at a target site. Cleavable linkers include, in some embodiments, enzymatically-cleavable peptide linkers, acid sensitive hydrazine linkers, and glutathione-sensitive disulfide linkers. Non-cleavable likers include, but are not limited to sulfosuccinimidyl 4-(N-maleimidomethyl)cyclohexane-1-carboxylate (SMCC).

In embodiments wherein the conjugate comprises a leukocyte-targeting molecule and the active pharmaceutical agent comprises a protein, the two amino acid sequences can be joined by a short peptide linker which is cleavable or non-cleavable.

In some embodiments, the linker covalently linking the one or more active pharmaceutical agent to the peptide compound comprises a structure selected from:

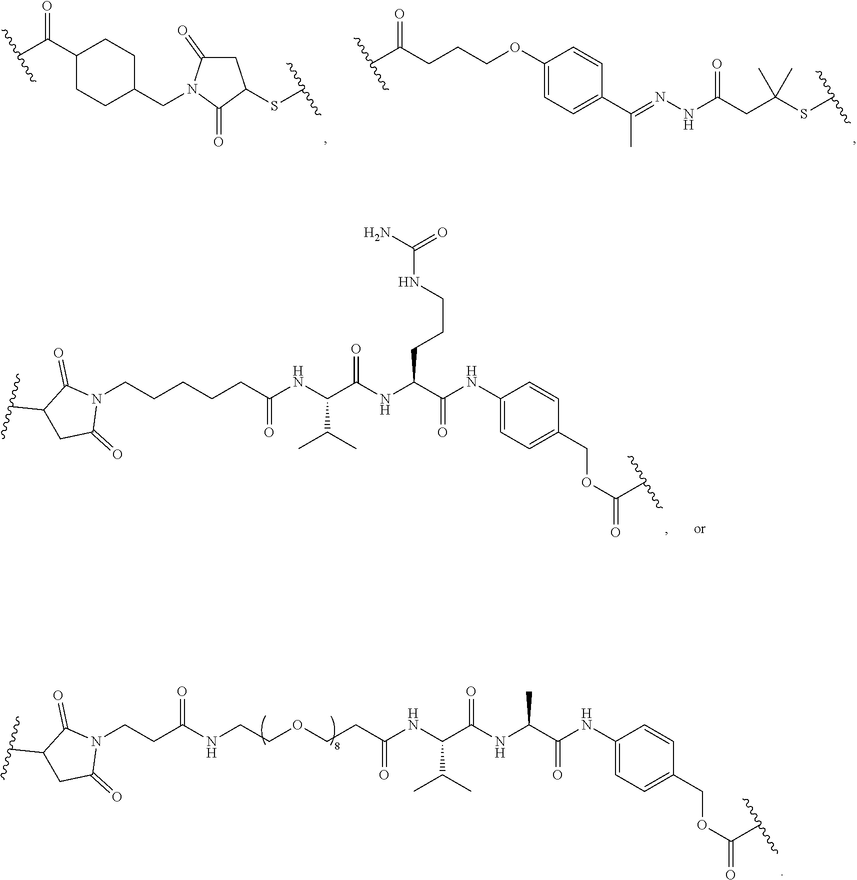

In some embodiments, the active pharmaceutical agent linked to the leukocyte-targeting molecule is selected from a peptide, a protein, a small molecule therapeutic, an oligomer (e.g., an oligonucleotide, e.g., an RNA or a DNA), or a radionucleotide, or a combination thereof. In some embodiments, the active pharmaceutical agent is methotrexate, which is useful in treating a variety of diseases, including treating cancers, for example, leukemias, breast cancer, skin cancer, head and neck cancer, lung cancer, uterus cancer, psoriasis, rheumatoid arthritis, atopic dermatitis, autoimmune diseases, and inflammatory diseases.

In some embodiments, the active pharmaceutical agent, and thereby a conjugate comprising such herein, is useful in treating acute lung inflammation, a liver disease, hypercholesterolemia, atherosclerosis, fatty liver, diabetes type 1, a skin disease or disorder (for example topically induced inflammation or burn), or a sepsis (for example, polymicrobial sepsis), alopecia areata, an autoimmune disease. In some embodiments, the inflammation or inflammatory disease refers to a disease or disorder including, but not limited to, acute disseminated encephalomyelitis (ADEM), Addison's disease, an allergy, allergic rhinitis, Alzheimer's disease, anti-phospholipid antibody syndrome (APS), an arthritis such as, for example, a monoarthritis, an oligoarthritis, or a polyarthritis like an osteoarthritis, a rheumatoid arthritis, a juvenile idiopathic arthritis, a septic arthritis, spondyloarthropathy, gout, pseudogout, Still's disease, asthma, autoimmune hemolytic anemia, autoimmune hepatitis, an autoimmune inner ear disease, bullous pemphigoid, celiac disease, Chagas disease, chronic obstructive pulmonary disease (COPD), diabetes mellitus type 1 (IDDM), endometriosis, a gastrointestinal disorder such as, for example, an irritable bowel disease or an inflammatory bowel disease like Crohn's disease or an ulcerative colitis, Goodpasture's syndrome, Graves' disease, Guillain-Barré syndrome (GBS), Hashimoto's thyroiditis, hidradenitis suppurativa, idiopathic thrombocytopenia purpura, interstitial cystitis, a lupus, such as, for example, a discoid lupus erythematosus, a drug-induced lupus erythematosus. a lupus nephritis, a neonatal lupus, a subacute cutaneous lupus erythematosus, a chronic cutaneous lupus, or a systemic lupus erythematosus, morphea, multiple sclerosis (MS), myasthenia gravis, a myopathy such as, for example, a dermatomyositis, an inclusion body myositis, or a polymyositis, myositis, narcolepsy, neuromyotonia, Parkinson's disease, pemphigus vulgaris, pernicious anaemia, primary biliary cirrhosis, psoriasis, recurrent disseminated encephalomyelitis, rheumatic fever, scleroderma, Sjögren's syndrome, a skin disorder such as, for example, dermatitis, eczema, statis dermatitis, atopic dermatitis, hidradenitis suppurativa, psoriasis, rosacea, acne, or scleroderma, tenosynovitis, uveitis, vasculitis such as, for example, Buerger's disease, cerebral vasculitis, Churg-Strauss arteritis, cryoglobulinemia, essential cryoglobulinemic vasculitis, giant cell arteritis, Golfer's vasculitis, Henoch-Schonlein purpura, hypersensitivity vasculitis, Kawasaki disease, microscopic polyarteritis/polyangiitis, polyarteritis nodosa, polymyalgia rheumatica (PMR), rheumatoid vasculitis, Takayasu arteritis, Wegener's granulomatosis, or vitiligo. In some embodiments, the active pharmaceutical agent is useful in treating a viral disease, for example shingles, herpes simplex type 1 infection, herpes simplex type 2 infection, or severe acute respiratory syndrome coronavirus 2 (SARS-CoV-2) infection.

In some embodiments, the pharmaceutical agent comprises a peptide, a protein, a nucleic acid, a small molecule, a heavy metal, an imaging agent, or a radioactive agent, or a combination thereof. In some embodiments, the active pharmaceutical agent is radiolabeled, for example with a radioactive agent. In some embodiments, the pharmaceutical agent is an enzyme comprising, but not limited to, horseradish peroxidase or alkaline phosphatase. In some embodiments, the pharmaceutical agent is a nuclease such as an RNase. In some embodiments, the heavy metal comprises, but is not limited to, colloidal gold or gadolinium. In some embodiments, the radioactive agent comprises, but is not limited to, ³H, ¹⁴C, ¹²⁵I, ¹⁸F, or ¹³¹I. In some embodiments, the imaging agent is a radioactive agent or a fluorescent label. In some embodiments, the fluorescent label comprises, but is not limited to, fluorescein, rhodamine, or green fluorescent protein, or derivatives thereof.

In some embodiments, the pharmaceutical agent is a small molecule, for example a cancer chemotherapeutic agent. Non-limiting examples of chemotherapeutic agents include alkylating agents (including, but not limited to, altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, or trabectedin), nitrosoureas (including, but not limited to, carmustine, lomustine, or streptozocin), antimetabolites (including, but not limited to, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, trifluridine, or tipiracil), anthracyclines (including, but not limited to, daunorubicin, doxorubicin (adriamycin), epirubicin, idarubicin, or valrubicin), non-anthracycline anti-tumor antibiotics (including, but not limited to, bleomycin, dactinomycin, mitomycin-C, or mitoxantrone), topoisomerase inhibitors (including, but not limited to, Irinotecan, topotecan, etoposide, mitoxantrone, or teniposide), mitotic inhibitors (including, but not limited to, cabazitaxel, docetaxel, nab-paclitaxel, paclitaxel, vinblastine, vincristine, or vinorelbine), corticosteroids (including, but not limited to, prednisone, methylprednisolone, or dexamethasone), and other chemotherapeutic agents (including, but not limited to, all-trans-retinoic acid, arsenic trioxide, asparaginase, eribulin, hydroxyurea, ixabepilone, mitotane, omacetaxine, pegaspargase, procarbazine, romidepsin, or vorinostat).

Other examples of pharmaceutical agents which can be linked to the leukocyte-targeting molecules include, but are not limited to, antibiotics, corticosteroids, antiviral agents, anti-proliferative agents, immunosuppressive agents, or immunostimulatory agents.

Additional examples of pharmaceutical agents which can be linked to the leukocyte-targeting molecules are protein and peptide pharmaceutical agents, for example antibodies.

Further examples of pharmaceutical agents which can be linked to the leukocyte-targeting molecules disclosed herein are nucleic acid molecules. Exemplary nucleic acid molecules include, but are not limited to, deoxynucleic acids (DNA) and ribonucleic acids (RNA). Exemplary DNA therapeutics include, but are not limited to, antisense oligonucleotides, DNA aptamers and gene therapies. Exemplary RNA therapeutics include, but are not limited to, micro RNAs, short interfering RNAs, ribozymes, RNA decoys, and circular RNA.

In some embodiments, the conjugate has the formula:

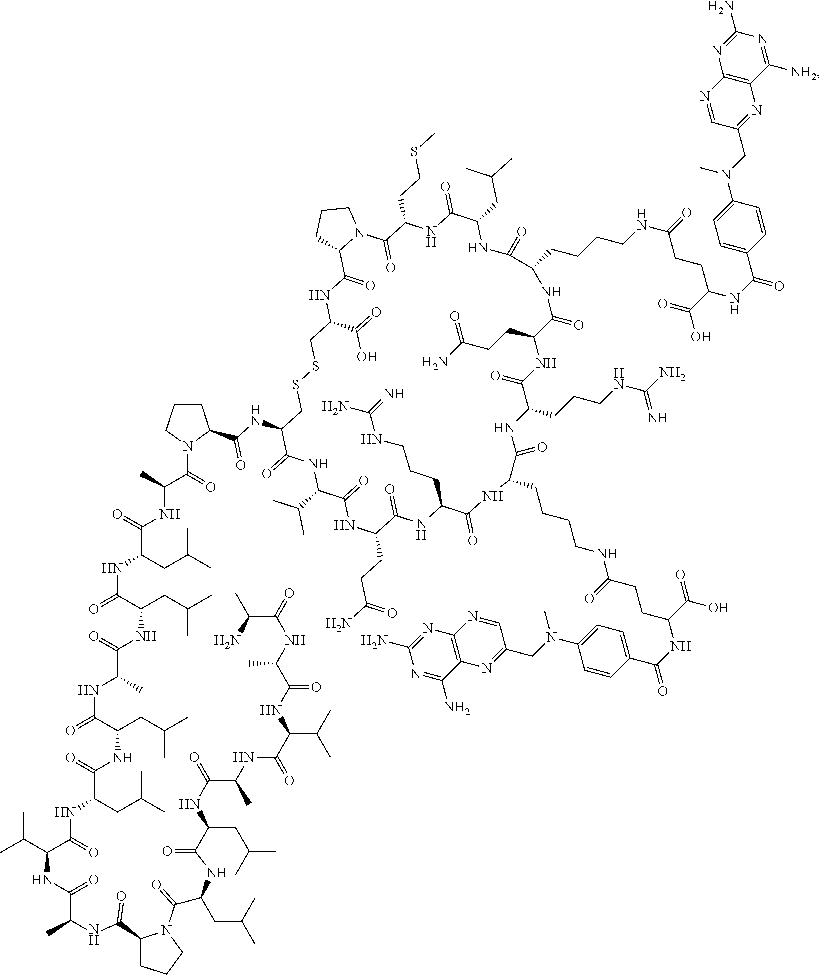

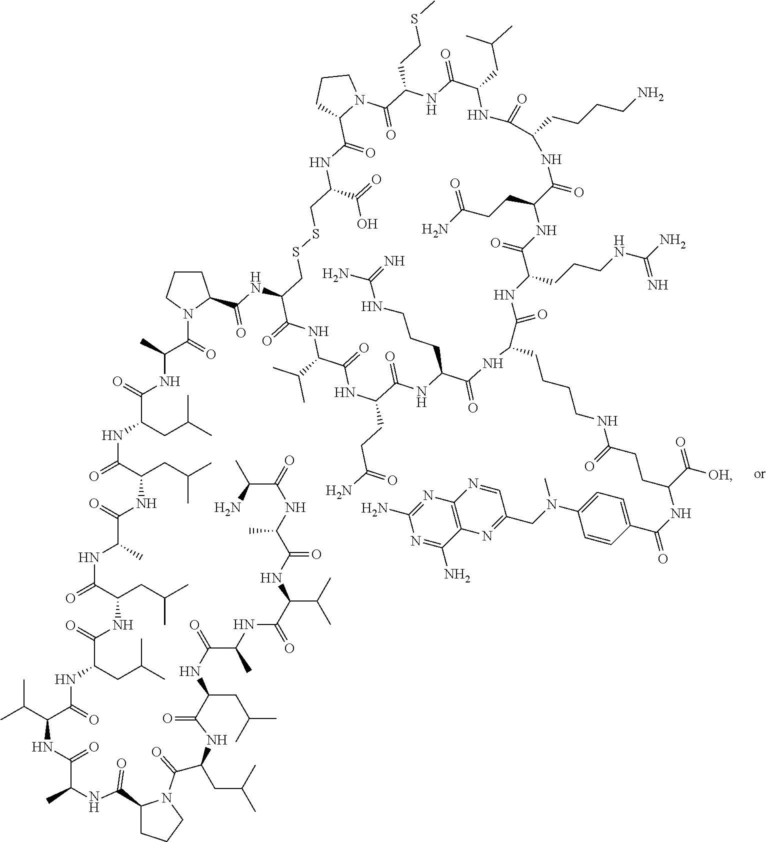

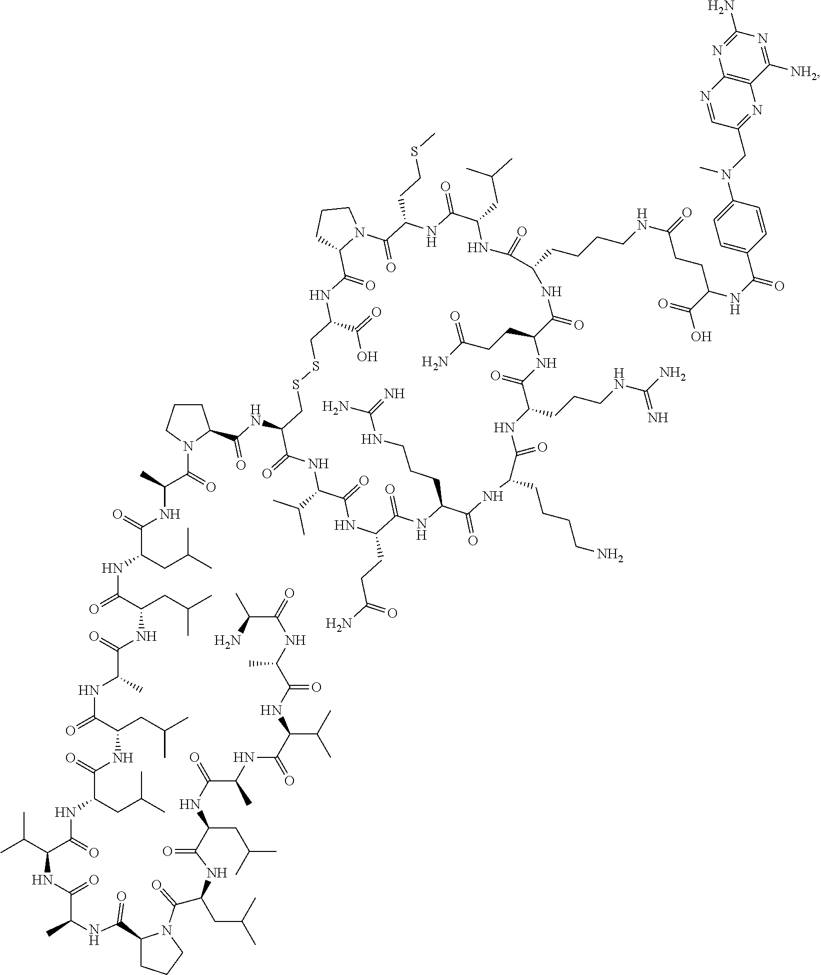

or a pharmaceutically acceptable salt thereof. In some embodiments, the conjugate is in a salt form. In some embodiments, the salt form of the conjugate includes at least one acetate counterion.

In some embodiments, the conjugate has the formula:

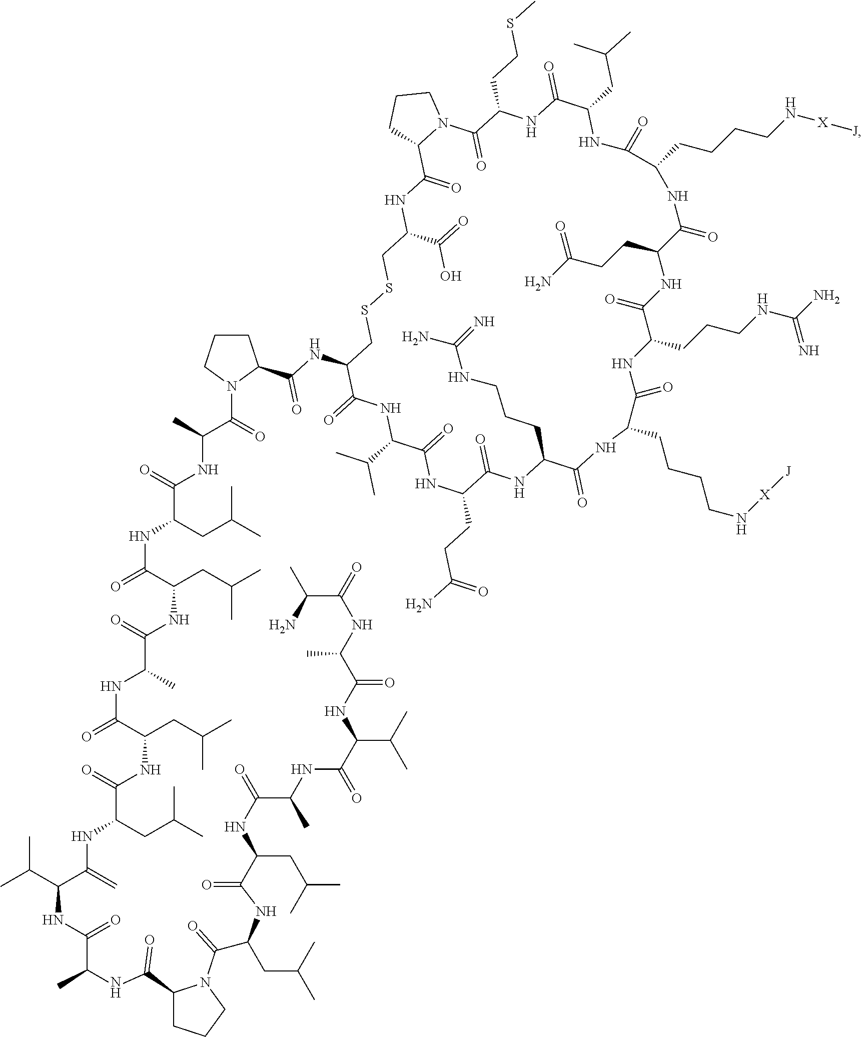

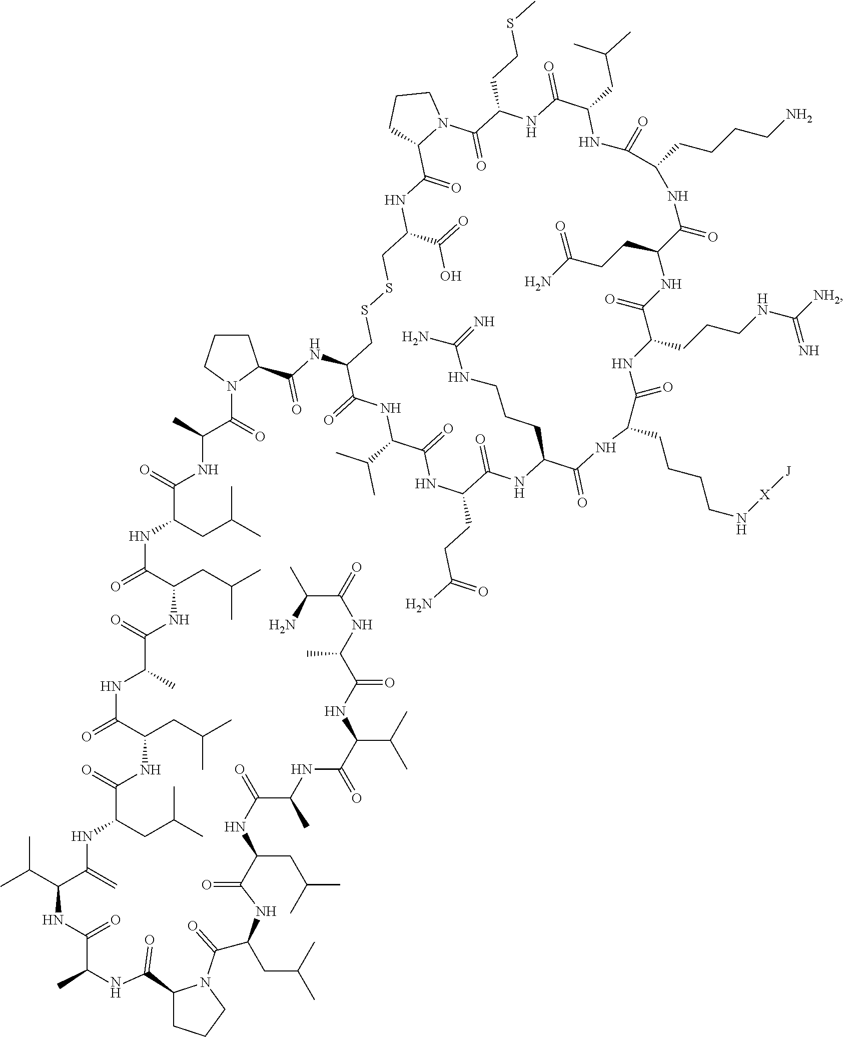

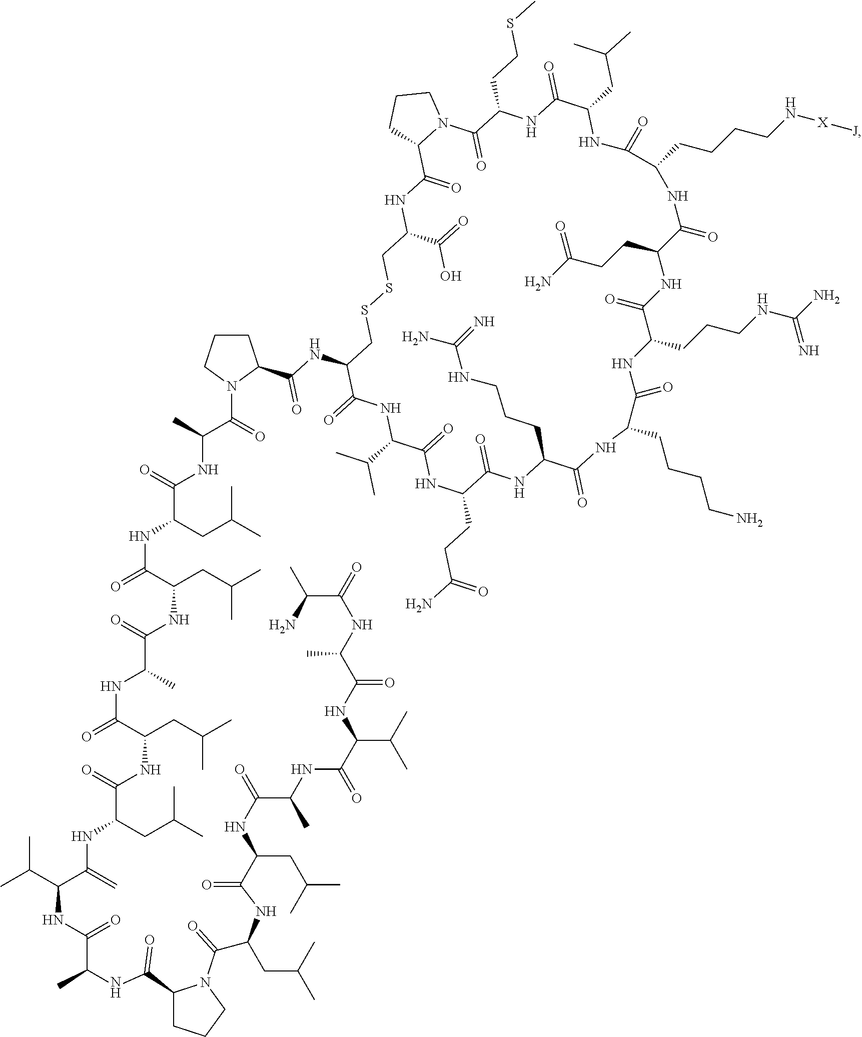

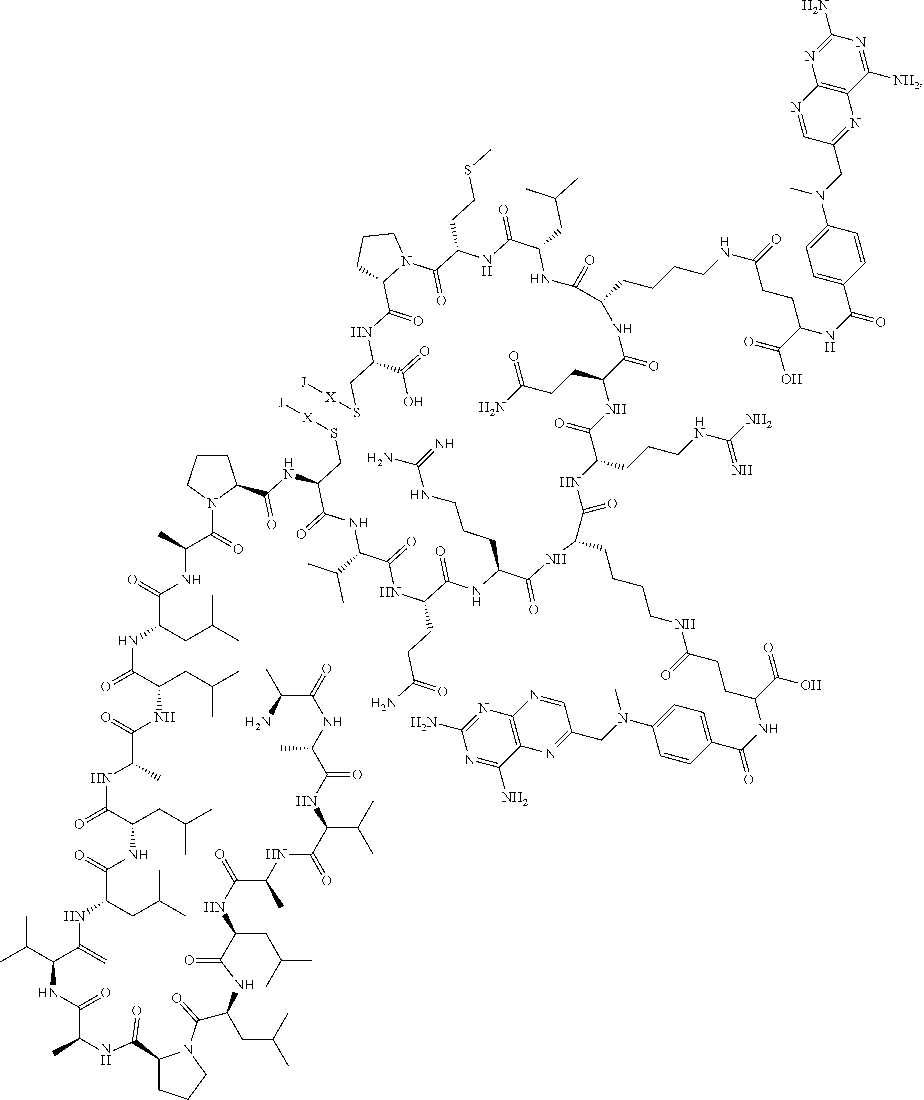

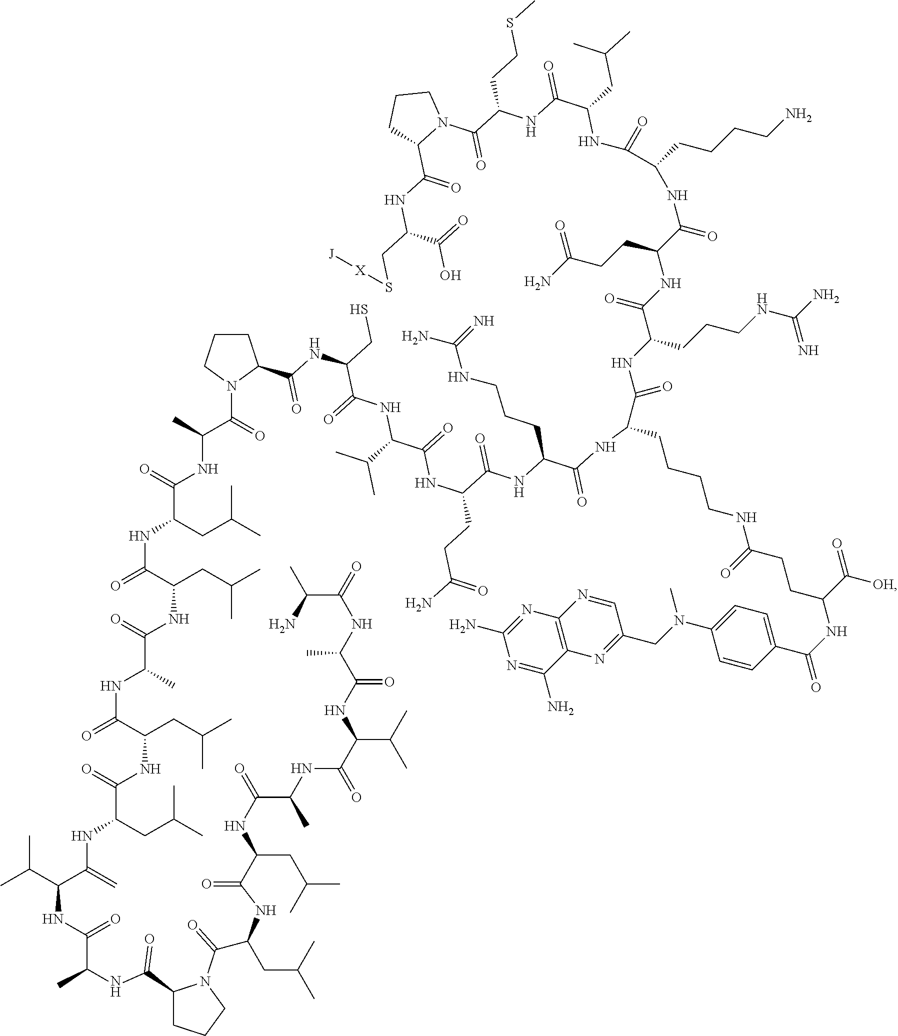

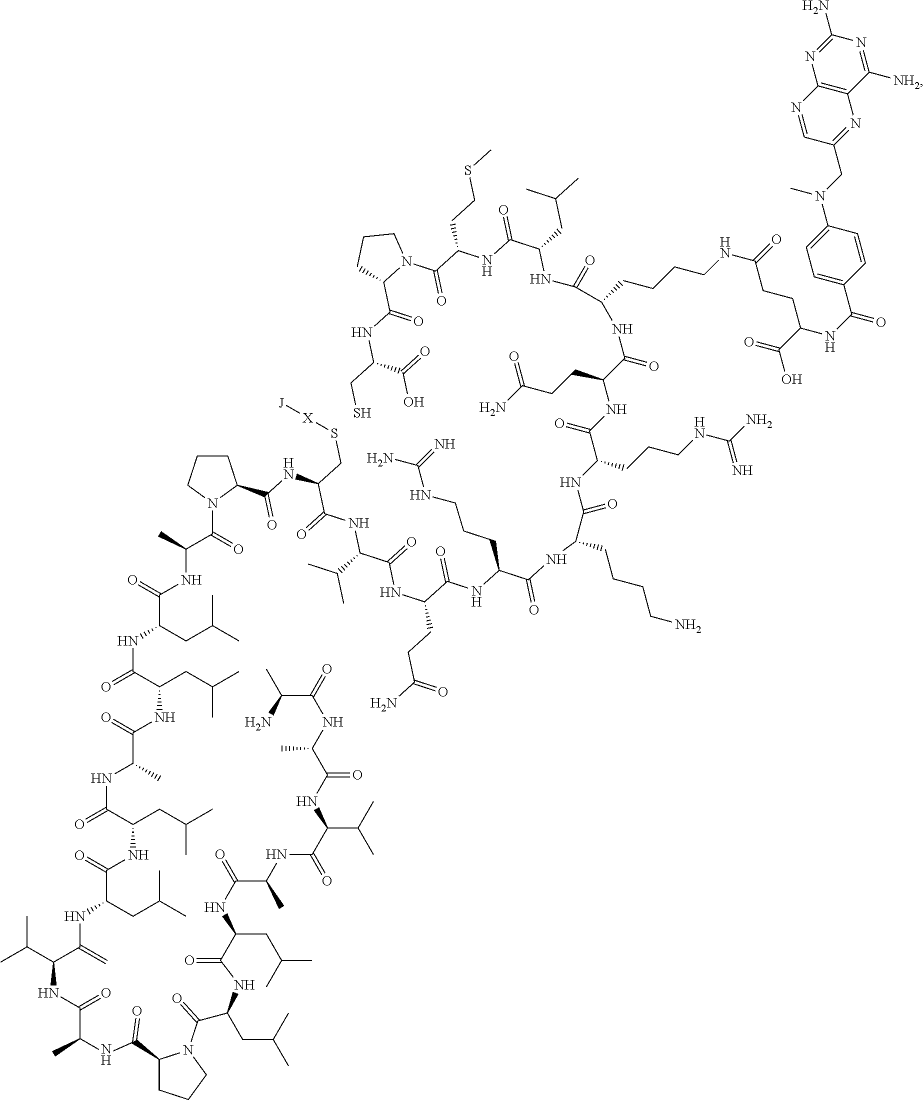

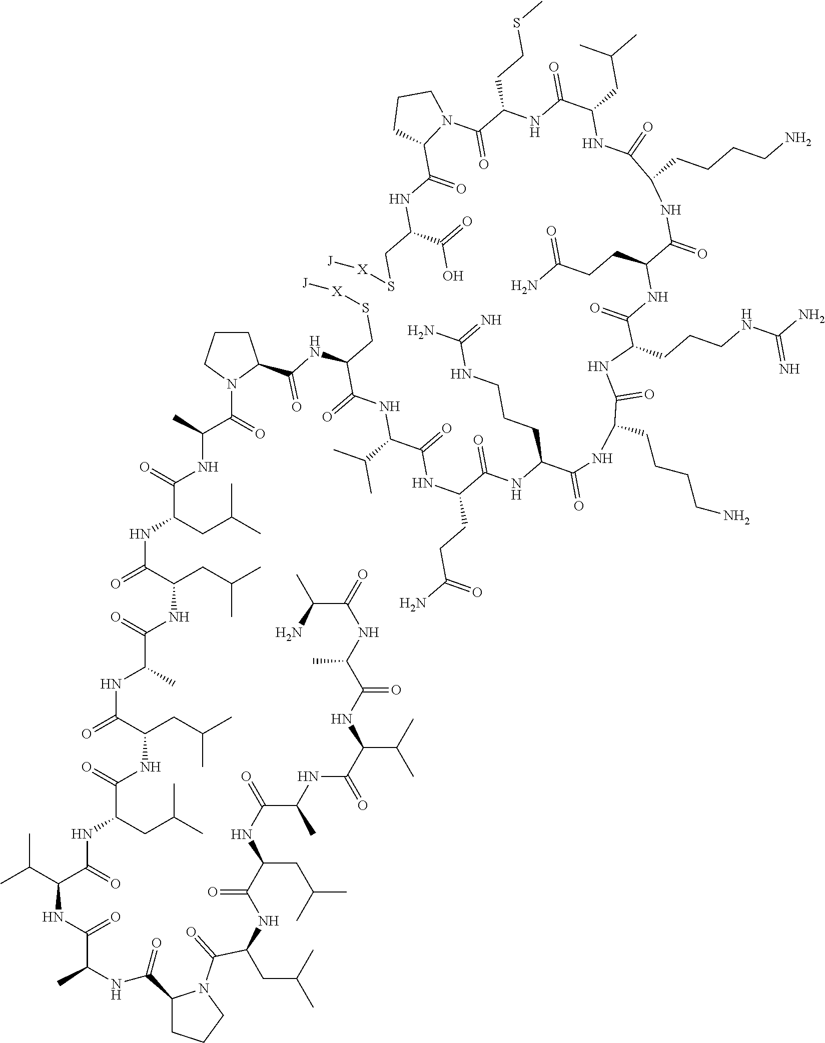

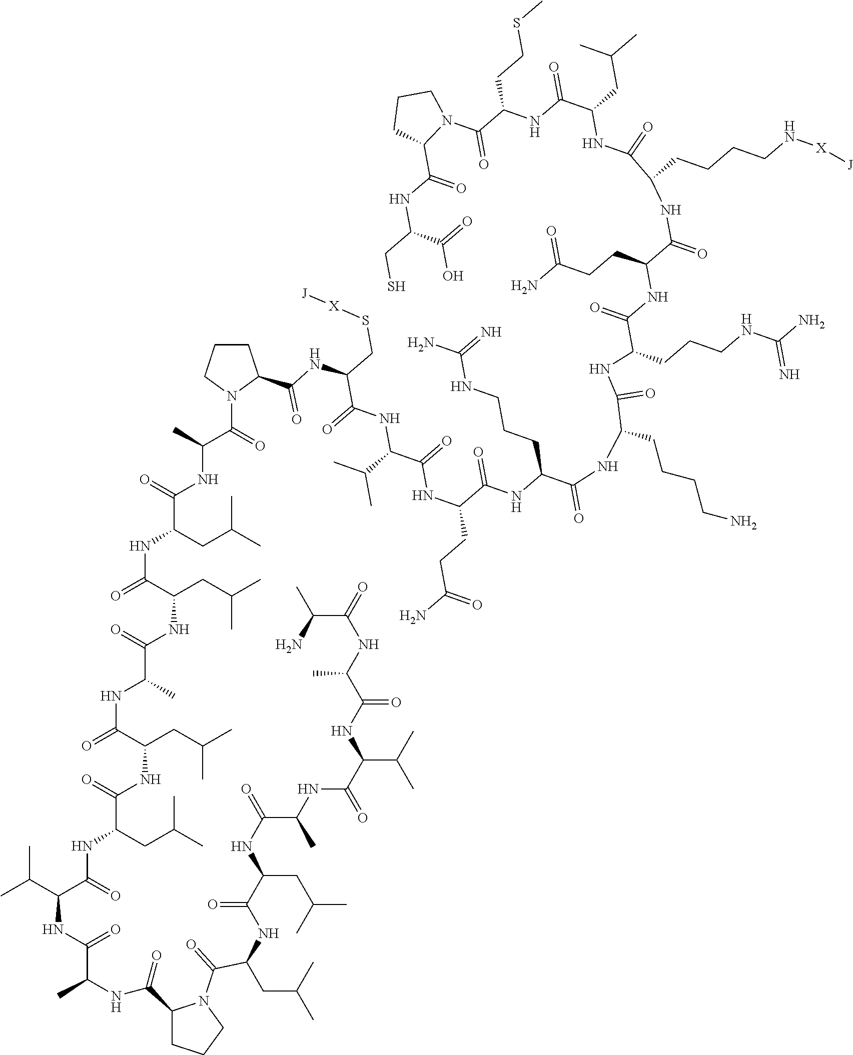

or a pharmaceutically acceptable salt thereof, wherein each J¹ is, independently, H or X-J, wherein one, two, or three of J¹ is H, and each X is, independently, a direct bond or a linker, and each J comprises, independently, an active pharmaceutical agent, including, without limitation, a nuclease, e.g., a ribonuclease or a deoxyribonuclease. In some embodiments, the conjugate is in a salt form. In some embodiments, the salt form of the conjugate includes at least one acetate counterion. In some embodiments of the compounds herein, each J is, independently, a ribonuclease.

In some embodiments, conjugates described herein also include isotopically-labeled conjugates wherein one or more atoms is replaced by an atom having the same atomic number, but an atomic mass or mass number different from the atomic mass or mass number predominantly found in nature. Examples of isotopes suitable for inclusion in include and are not limited to ²H, ³H, ¹¹C, ²³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In some embodiments, isotopically-labeled conjugates are useful in drug or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled conjugates are prepared by any suitable method or by processes using an appropriate isotopically-labeled reagent in place of the non-labeled reagent otherwise employed.

In some embodiments, the conjugates described herein are labeled by other means, including, but not limited to, the use of chromophores or fluorescent moieties, bioluminescent labels, chemiluminescent labels, or enzymes.

The conjugates described herein, and other related compounds having different substituents may be synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Conjugates described herein are synthesized using any suitable procedures starting from compounds that are available from commercial sources, or are prepared using procedures described herein.

In some embodiments, the compounds described herein may be prepared by a method of synthesis that comprises any of the synthetic schemes shown in FIG. 1.

In some embodiments, reactive functional groups, such as hydroxyl, amino, imino, thio, or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

In some embodiments, protective groups are removed by acid, base, reducing conditions (for example, by hydrogenolysis), or oxidative conditions. Groups such as trityl, dimethoxytrityl, acetal, and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

### Compositions

In some embodiments, provided herein are compositions comprising one or more conjugates described herein. In some embodiments, the composition is a pharmaceutical composition further comprising a pharmaceutically acceptable carrier.

Articles of manufacture are described, which comprise the conjugates provided herein. The articles of manufacture may include forms of the conjugates suitable for administration or storage.

The present conjugates and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (e.g., Irgacure 184, 2959), preferably water-soluble initiators (e.g., Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (e.g., by the addition of specific product information leaflets) into suitable containers (boxes, etc.).

According to further embodiments, the present conjugates can also be provided in kit form combined with other components necessary for administration of the material to the patient. For example, disclosed kits, such as for use in the treatment of leukocyte associated diseases, can further comprise, for example, administration materials. The kits are designed in various forms based on the specific deficiencies they are designed to treat.

The conjugates provided herein may be prepared and placed in a container for storage at ambient or elevated temperature. When the conjugate is stored in a polyolefin plastic container, for example, as compared to a polyvinyl chloride plastic container, discoloration of the conjugate or adsorption of the conjugate may be reduced. Without wishing to be bound by theory, the container may reduce exposure of the container's contents to electromagnetic radiation, whether visible light (e.g., having a wavelength of about 380-780 nm) or ultraviolet (UV) light (e.g., having a wavelength of about 190-320 nm (UV B light) or about 320-380 nm (UV A light)). Some containers also include the capacity to reduce exposure of the container's contents to infrared light, or a second component with such a capacity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a bag, tube, tub, or bottle. In some embodiments, the container is a glass container. The container may further be disposed within a second container, for example, a paper, cardboard, paperboard, metallic film, or foil, or a combination thereof, container to further reduce exposure of the container's contents to UV, visible, or infrared light. Conjugates benefiting from reduced discoloration, decomposition, or both during storage, include the conjugates provided herein. The conjugates or compositions provided herein may need storage lasting up to, or longer than, three months; in some cases up to, or longer than one year. The containers may be in any form suitable to contain the contents; for example, a bag, a bottle, a tube, a tub, or a box, or a combination thereof.

In some embodiments, provided herein are packaged conjugates, or packaged pharmaceutical compositions, e.g., kits, comprising a container housing an effective amount of a conjugate in a composition described herein, and instructions for using the composition in accordance with one or more of the methods provided herein.

### Methods

In some embodiments, the conjugates provided herein are useful in treating leukocyte-associated diseases on administration of a conjugate provided herein to a subject in need thereof.

In some embodiments, provided herein are methods of treating a cancer or an inflammatory disease in a subject in need thereof, comprising administering a therapeutically effective amount of a conjugate provided herein to the subject.

In some embodiments, the conjugates provided herein are useful for targeting of diagnostic, therapeutic, and prophylactic agents to leukocytes. In some embodiments, the leukocytes are targeted in a subject in need of treatment for a disease or disorder associated with a known use of the active pharmaceutical agent conjugated to the leukocyte-targeting moiety.

In some embodiments, provided herein are methods of delivering an active pharmaceutical agent of a conjugate provided herein to a white blood cell or a CD34+ stem cell in a subject in need thereof, comprising administering a conjugate provided herein to the subject. In some embodiments, delivery of the conjugate or active pharmaceutical agent to the white blood cell or the CD34+ stem cell occurs within about 2 hours or less from the time of administering the formulation.

In some embodiments of the methods provided herein, the active pharmaceutical agent or conjugate is delivered preferentially to white blood cells and CD34+ stem cells over red blood cells.

In some embodiments of these methods, the conjugate may be provided in a dosage form that is suitable for local or systemic delivery. Routes of administration include, without limitation, intravenous, oral, nasal, rectal, intravaginal, parenteral, buccal, sublingual, or topical. In some embodiments, the oral or nasal route of administration is an oral inhalational or nasal inhalational route of administration. The conjugates for use as described herein may be formulated for administration by any suitable route to achieve the particular method being applied.

In some embodiments, the conjugates may be formulated as a cream formulation for topical administration.

The compositions provided herein may be prepared according to conventional pharmaceutical practice (see, e.g., (Gennaro, A. R. ed. (2000) Remington: The Science and Practice of Pharmacy (20th ed.), Lippincott Williams & Wilkins, Baltimore, Md.; Swarbrick, J. and Boylan, J. C. eds. (1988-1999) Encyclopedia of Pharmaceutical Technology, Marcel Dekker, New York). The leukocyte-targeting molecules described herein may be prepared analogous to methods described in U.S. Pat. No. 8,324,148B2, the entire content of which is incorporated by reference herein.

The therapeutic methods described herein in general include administration of a therapeutically effective amount of a conjugate described herein to a subject (e.g., animal) in need thereof, including a mammal. In some embodiments, the subject is a human. In some embodiments, the subject is a non-human mammal. Such treatment will be suitably administered to subjects in need thereof. Determination of those subjects “in need thereof” can be made by any objective or subjective determination by a diagnostic test or opinion of a subject or health care provider.

Actual dosage levels of the active ingredients (e.g., the conjugates described herein), the formulations, or the pharmaceutical compositions provided herein may be varied so as to obtain an amount of the active ingredient that is effective to achieve the desired therapeutic response for a particular patient, formulation, and mode of administration, without being toxic to the patient. In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health, and prior medical history of the patient being treated, and like factors well-known in the medical arts. A medical doctor, e.g., physician or veterinarian, having ordinary skill in the art may readily determine and prescribe the effective amount of the pharmaceutical composition required. For example, the physician or veterinarian could start doses of the conjugates employed in the pharmaceutical composition at levels lower than that required in order to achieve the desired therapeutic effect and gradually increase the dosage until the desired effect is achieved.

The following examples further illustrate aspects of the present disclosure. However, they are in no way a limitation of the teachings or disclosure as described herein.

### EXAMPLES

### Example 1

A conjugate comprising methotrexate is prepared as shown in FIG. 1 or FIG. 2. The conjugation to methotrexate was confirmed by HPLC and Mass Spec analyses.

### Example 2

A conjugate comprising methotrexate is administered to a subject suffering from a leukemia. A clinical reduction in the severity of the subject's leukemia is observed.

### Example 3

### Conjugation of SEQ ID NO: 14 to an RNase.

SEQ ID NO: 14 is conjugated to an RNase, and SEQ ID NO: 14's targeting ability is used to localize to leucocytes, enter the cell, and selectively kill these cells by the RNase activity. A Controlled Protein-Protein Crosslinking Kit from Thermo Scientific (Number 23456) is used. HPLC data shows that a conjugate is produced according to the following protocol.

The Controlled Protein-Protein Crosslinking Kit provides a way to couple any two proteins through an amine (—NH2) functional group on one protein and a sulfhydryl (—SH) group on the other using Sulfo-SMCC as the crosslinking agent. Sulfo-SMCC is a heterobifunctional crosslinker that contains an N-hydroxysuccinimide (NHS) ester and a maleimide group. NHS esters react with primary amines at pH 7-9 to form covalent amide bonds. Maleimides react with sulfhydryl groups at pH 6.5-7.5 to form stable thioether bonds.

The maleamide group of Sulfo-SMCC reacts with RNase, and the sulfo group reacts with free thiols on SEQ ID NO: 14 to produce a stable linkage.

The RNAse is activated with the Sulfo-SMCC. hRNase wt FC, 2.76 mg/mL reacts with the Sulfo-SMCC, and then purified using a gel-filtration column.

The disulfide bonds of SEQ ID NO: 14 are reduced to create free SH-groups.

Maleimide-RNase and sulfhydryl-SEQ ID NO: 14 are mixed in approximately equal molar amounts. Generally, if the concentrations of the proteins are approximately equal, reacting equal mass amounts of the two proteins will achieve sufficient crosslinking. The success of the coupling is verified by HPLC analysis.

### Example 4

### Conjugation of a Leukocyte-Targeting Molecule/Compound to a Protein.

The foregoing conjugation protocol is used generally, with or without minor modifications, to conjugate a leukocyte-targeting molecule/compound with an active pharmaceutical agent, such as a protein.

### Example 5

### Treatments.

A conjugate prepared as in Example 1, Example 3, or Example 4 is administered to a subject suffering from a leukocyte-associated disease, such as a cancer, psoriasis, rheumatoid arthritis, atopic dermatitis, an autoimmune disease, or an inflammatory disease. A clinical reduction in the severity of the subject's leukocyte-associated disease is observed.

We claim:

1. A compound, having a formula:
Z—(X-J)ₙ
or a pharmaceutically acceptable salt thereof,
wherein:
Z is a leukocyte-targeting molecule;
X is, independently, a direct bond or a linker;
J is, independently, an active pharmaceutical agent; and
n is 1, 2, 3, or 4, or more than 4.

2. The compound of claim 1, wherein the leukocyte-targeting molecule comprises an amino acid sequence selected from:
(SEQ ID NO: 1)
AVALLPAVLLALLA;
(SEQ ID NO: 2)
AAVAPAVX¹⁶X¹⁶AX¹⁶X¹⁶A;
(SEQ ID NO: 3)
AAVALLPAVLLALLA;
(SEQ ID NO: 4)
X¹X¹AAVALLPAVLLALLAX¹X¹;
(SEQ ID NO: 5)
KKAAVALLPAVLLALLAKK;
(SEQ ID NO: 6)
RRAAVALLPAVLLALLARR;
(SEQ ID NO: 7)
RRAAVALLPAVLLALLARK;
(SEQ ID NO: 8)
RKAAVALLPAVLLALLARKY;
(SEQ ID NO: 9)
AAVALLPAVLLALLAPCVQRKRQKLMPC;
(SEQ ID NO: 10)
X¹X²AAX³AX⁴X⁵X¹⁷AX⁶X7X⁸AX⁹X¹⁰A(P)ₙX¹¹X¹²(X¹³)ₙ;
(SEQ ID NO: 11)
X²X²AAVALLPAVLLALLAPX¹¹X¹²(X¹³)ₙ;
(SEQ ID NO: 12)
X¹X²AAX³AX⁴X⁵X¹⁷AX⁶X7X⁸AX⁹X¹⁰APX¹¹X¹²(X¹³)ₙCVQRKRQKLMP
C;
(SEQ ID NO: 13)
AAVALLPAVLLALLAPVQRKRQKLMP;
(SEQ ID NO: 14)
AAVALLPAVLLALLAPCVQRKRQKLMPC (cyclized by SS
bond at C¹⁷ and C²⁸);
(SEQ ID NO: 15)
AAVAPAVX¹⁶X¹⁶AX¹⁶X¹⁶AP;
(SEQ ID NO: 16)
AAVALLPAVLLALLAP;
(SEQ ID NO: 17)
(X¹⁴)ₙ(X¹⁵)ₙAAVALLP(X¹⁴)ₘ;
(SEQ ID NO: 18)
(X¹⁴)ₙ(X¹⁵)ₙAVLLALLAP(X¹⁴)ₘ;
(SEQ ID NO: 19)
(X¹⁴)ₙ(X¹⁵)ₙAAVALLAAVLLALLAP(X¹⁴)ₘ;
(SEQ ID NO: 20)
TTGTLLPGVLLALVVA;
(SEQ ID NO: 21)
(X¹⁴)ₙ(X¹⁵)ₙTTGTLLPGVLLALVVA(X¹⁴)ₘ;
(SEQ ID NO: 22)
TTGTLLPRVLLALVVA;
(SEQ ID NO: 23)
(X¹⁴)ₙ(X¹⁵)ₙTTGTLLPGVLLALVVA(X¹⁴)ₘ;
(SEQ ID NO: 24)
(X¹⁴)ₙ(X¹⁵)ₙTTGTLLP(X¹⁴)ₘ;
(SEQ ID NO: 25)
(X¹⁴)ₙ(X¹⁵)ₙVLLALVVA(X¹⁴)ₘ;
(SEQ ID NO: 26)
RAAVALLPAVLLALLAPY(X¹⁴)ₘ;
(SEQ ID NO: 27)
RAAVALLPAVLLAVLAPY(X¹⁴)ₘ;
(SEQ ID NO: 28)
(X¹⁴)ₙ(X¹⁵)ₙAAVALLPAVLLALLAPDVRKRQDLEQ KM(X¹⁴)_{z}(Y)ₘ;
(SEQ ID NO: 29)
(X¹⁴)ₙ(X¹⁵)ₙAAVALLPAVLLALLAP(X¹⁴)ₘ;
(SEQ ID NO: 30)
(X¹⁴)ₙ(X¹⁵)ₙAAVALLAAVLLALLAP(X¹⁴)ₘ;
(SEQ ID NO: 31)
(X¹⁴)ₙ(X¹⁵)ₙTTGTLLPGVLLALVVA(X¹⁴)ₘ;
(SEQ ID NO: 32)
(X¹⁴)ₙ(X¹⁵)ₙTTGTLLPRVLLALVVA(X¹⁴)ₘ;
(SEQ ID NO: 33)
(X¹⁴)ₙ(X¹⁵)ₙTTGTLLP(X¹⁴)ₘ;
(SEQ ID NO: 34)
(X¹⁴)ₙ(X¹⁵)ₙGVLLALVVA(X¹⁴)ₘ;
(SEQ ID NO: 35)
(X¹⁴)ₙ(X¹⁵)ₙAAVALLPAVLLALLAPCVQKRQKLMPC;
(SEQ ID NO: 36)
KKAAVALLPAVLLALLAPKK;
(SEQ ID NO: 37)
RRAAVALLPAVLLALLAPRR;
(SEQ ID NO: 38)
RRAAVALLPAVLLALLAPRK;
(SEQ ID NO: 39)
RKAAVALLPAVLLALLAPRKY;
(SEQ ID NO: 40)
KRAAVALLPKK;
(SEQ ID NO: 41)
KRAVLLALLAPKK;
(SEQ ID NO: 42)
KRAAVALLAAVLLALLAPKK;
(SEQ ID NO: 43)
KRTTGTLLPGVLLALVVAKK;
(SEQ ID NO: 44)
KRTTGTLLPGVLLALVVAKK;
(SEQ ID NO: 45)
KRTTGTLLPKK;
(SEQ ID NO: 46)
KRVLLALVVAKK;
(SEQ ID NO: 47)
KRAAVALLPKK;
(SEQ ID NO: 48)
AAVALLPAVLLALLAP;
(SEQ ID NO: 49)
AAVALLPAVLLALLAPCYVQRKRQKLMPC;
(SEQ ID NO: 50)
AAVALLPAVLLAVLAPCVQRKRQKLMPC;
(SEQ ID NO: 51)
AAVALLPAVLLALLAPCVQRDEQKLMPC;
(SEQ ID NO: 52)
AAVALLPAVLLAVLAPCVQRDEQKLMPC;
(SEQ ID NO: 53)
X¹X²AAX³AX⁴X⁵PAX⁶X⁷X⁸AX⁹X¹⁰A(P)ₘX¹¹X¹²(X¹³)ₙ;
(SEQ ID NO: 54)
LLAAVALLPAVLLALLA;
(SEQ ID NO: 55)
AAVALLPAVLLALLALL;
(SEQ ID NO: 56)
LLAAVALLPAVLLALLALL;
(SEQ ID NO: 57)
LLAAVALLPAVLLALLAP;
(SEQ ID NO: 58)
AAVALLPAVLLALLAPLL;
(SEQ ID NO: 59)
LLAAVALLPAVLLALLAPLL;
(SEQ ID NO: 60)
(X¹⁴)ₙAAVALLPAVLLALLA(X¹⁴)ₙ;
(SEQ ID NO: 61)
(X¹⁴)ₙAAVALLPAVLLALLAP(X¹⁴)ₙ;
(SEQ ID NO: 62)
(X¹⁴)ₙAAVALLPAVLLALLA(X¹⁴)ₙDVRKRQDLEQKMKKY;
(SEQ ID NO: 63)
(X¹⁴)ₙAAVALLPAVLLALLAP(X¹⁴)ₙDVRKRQDLEQKMKKY;
(SEQ ID NO: 64)
(X¹⁴)ₙAAVALLPAVLLALLA(X¹⁴)ₙKTGLRRLQHR;
(SEQ ID NO: 65)
(X¹⁴)ₙAAVALLPAVLLALLAP(X¹⁴)ₙKTGLRRLQHR;
(SEQ ID NO: 66)
AAVALLPAVLLALLADVRKRQDLEQKMKKY;
(SEQ ID NO: 67)
AAVALLPAVLLALLAPDVRKRADLEQKMKKY;
(SEQ ID NO: 68)
AAVALLPAVLLALLAKTGLRRLQHR;
(SEQ ID NO: 69)
AAVALLPAVLLALLAPKTGLRRLQHR;
(SEQ ID NO: 70)
CVQRKRQKLMPC;
(SEQ ID NO: 71)
X¹X²AAX³AX⁴X⁵PAX⁶X⁷X⁸AX⁹X¹⁰A(P)ₘX¹¹X¹²(X¹³)ₙCVQRKRQKLMP
C;
(SEQ ID NO: 72)
AAVALLPAVLLALLACVQRKRQKLMPC;
or a pharmaceutically acceptable salt thereof;
wherein
X¹, X², X¹¹, and X¹² are each, independently, lysine, arginine, or ornithine;
X³, X⁴, X⁵, X⁶, X⁷, X⁸, X⁹, and X¹⁰ are each, independently, valine, leucine, isoleucine, or norleucine;
X¹³ is tyrosine;
X¹⁴ is lysine;
X¹⁵ is arginine;
X¹⁶ is leucine, isoleucine, or norleucine;
X¹⁷ is proline or alanine;
n is 0, 1, 2, 3 or 4; and
m is 0, 1, 2, 3 or 4.

3. The compound of claim 1 or claim 2, wherein the leukocyte-targeting molecule comprises a disulfide bond between two cysteine residues.

4. The compound of one of claims 1-3, wherein the leukocyte-targeting molecule has a formula:

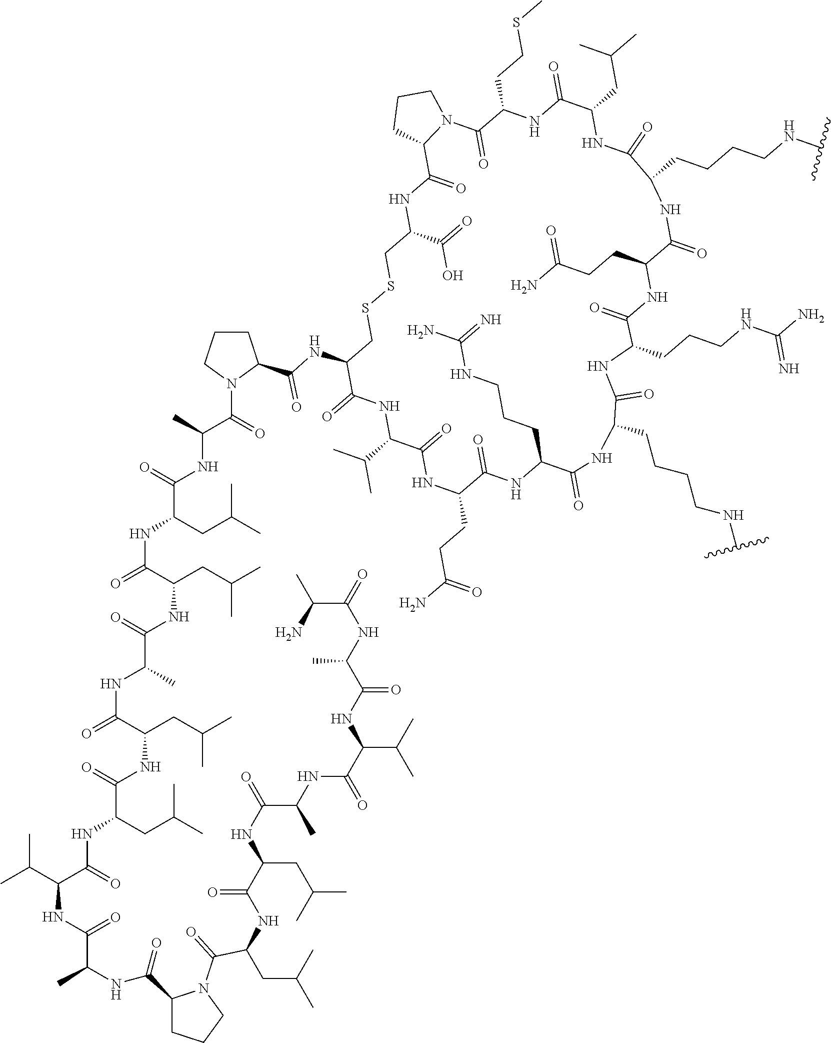 [SMILES: *NCCCC[C@@H]1NC(=O)[C@H](CCCNC(=N)N)NC(=O)[C@H](CCC(N)=O)NC(=O)[C@H](C(C)C)NC(=O)[C@@H](NC(=O)[C@@H]2CCCN2C(=O)[C@H](C)NC(=O)[C@H](CC(C)C)NC(=O)[C@H](CC(C)C)NC(=O)[C@H](C)NC(=O)[C@H](CC(C)C)NC(=O)[C@H](CC(C)C)NC(=O)[C@@H](NC(=O)[C@H](C)NC(=O)[C@@H]2CCCN2C(=O)[C@H](CC(C)C)NC(=O)[C@H](CC(C)C)NC(=O)[C@H](C)NC(=O)[C@@H](NC(=O)[C@H](C)NC(=O)[C@H](C)N)C(C)C)C(C)C)CSSC[C@@H](C(=O)O)NC(=O)[C@@H]2CCCN2C(=O)[C@H](CCSC)NC(=O)[C@H](CC(C)C)NC(=O)[C@H](CCCCN*)NC(=O)[C@H](CCC(N)=O)NC(=O)[C@H](CCCNC(=N)N)NC1=O]

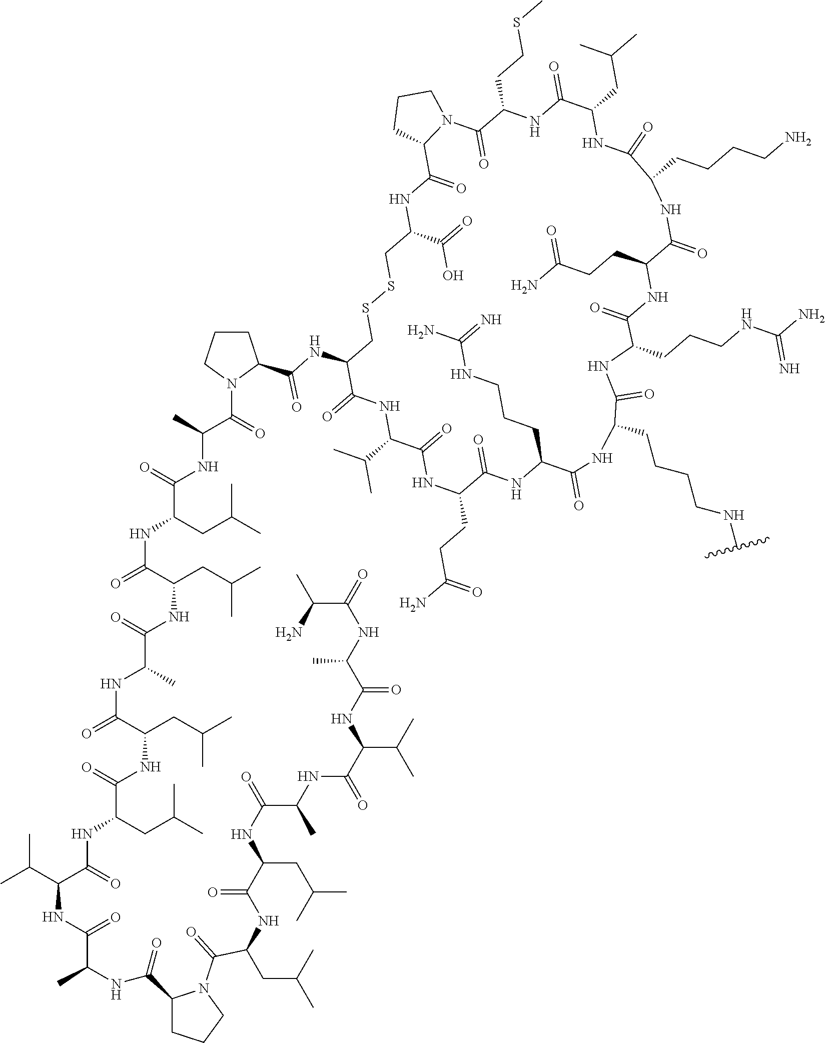 [SMILES: *NCCCC[C@@H]1NC(=O)[C@H](CCCNC(=N)N)NC(=O)[C@H](CCC(N)=O)NC(=O)[C@H](C(C)C)NC(=O)[C@@H](NC(=O)[C@@H]2CCCN2C(=O)[C@H](C)NC(=O)[C@H](CC(C)C)NC(=O)[C@H](CC(C)C)NC(=O)[C@H](C)NC(=O)[C@H](CC(C)C)NC(=O)[C@H](CC(C)C)NC(=O)[C@@H](NC(=O)[C@H](C)NC(=O)[C@@H]2CCCN2C(=O)[C@H](CC(C)C)NC(=O)[C@H](CC(C)C)NC(=O)[C@H](C)NC(=O)[C@@H](NC(=O)[C@H](C)NC(=O)[C@H](C)N)C(C)C)C(C)C)CSSC[C@@H](C(=O)O)NC(=O)[C@@H]2CCCN2C(=O)[C@H](CCSC)NC(=O)[C@H](CC(C)C)NC(=O)[C@H](CCCCN)NC(=O)[C@H](CCC(N)=O)NC(=O)[C@H](CCCNC(=N)N)NC1=O]

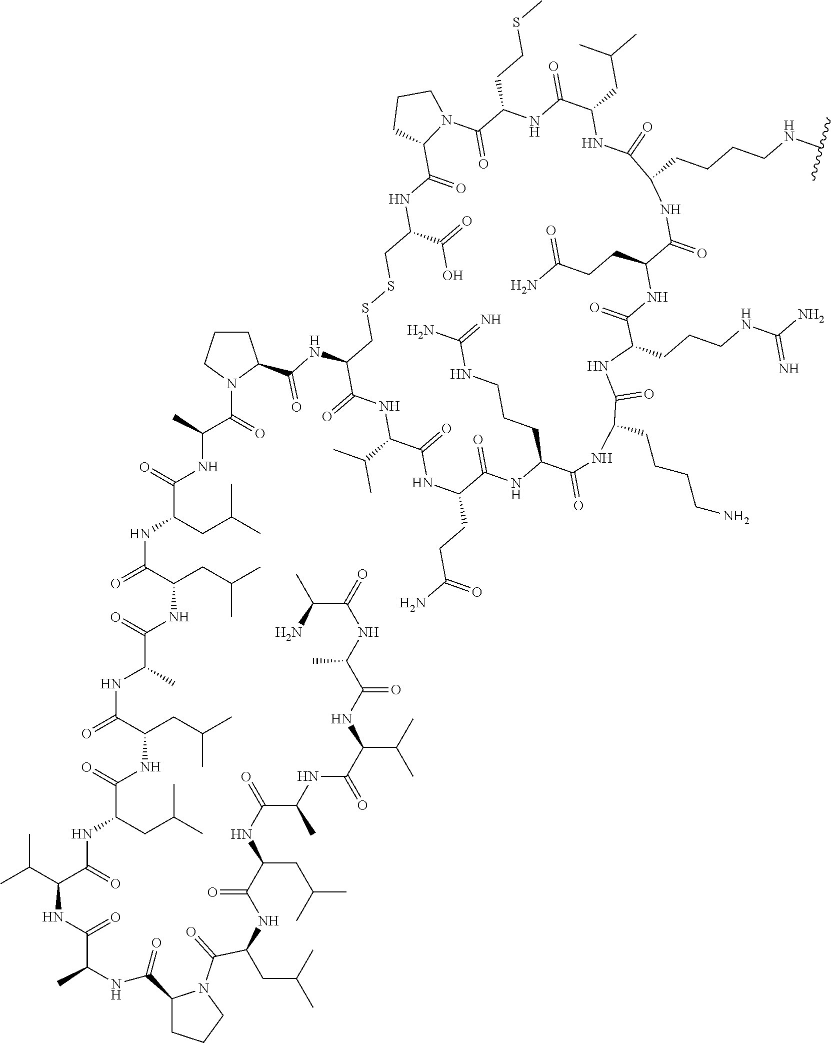 [SMILES: *NCCCC[C@@H]1NC(=O)[C@H](CCC(N)=O)NC(=O)[C@H](CCCNC(=N)N)NC(=O)[C@H](CCCCN)NC(=O)[C@H](CCCNC(=N)N)NC(=O)[C@H](CCC(N)=O)NC(=O)[C@H](C(C)C)NC(=O)[C@@H](NC(=O)[C@@H]2CCCN2C(=O)[C@H](C)NC(=O)[C@H](CC(C)C)NC(=O)[C@H](CC(C)C)NC(=O)[C@H](C)NC(=O)[C@H](CC(C)C)NC(=O)[C@H](CC(C)C)NC(=O)[C@@H](NC(=O)[C@H](C)NC(=O)[C@@H]2CCCN2C(=O)[C@H](CC(C)C)NC(=O)[C@H](CC(C)C)NC(=O)[C@H](C)NC(=O)[C@@H](NC(=O)[C@H](C)NC(=O)[C@H](C)N)C(C)C)C(C)C)CSSC[C@@H](C(=O)O)NC(=O)[C@@H]2CCCN2C(=O)[C@H](CCSC)NC(=O)[C@H](CC(C)C)NC1=O]

5. The compound of claim 1, having a formula: 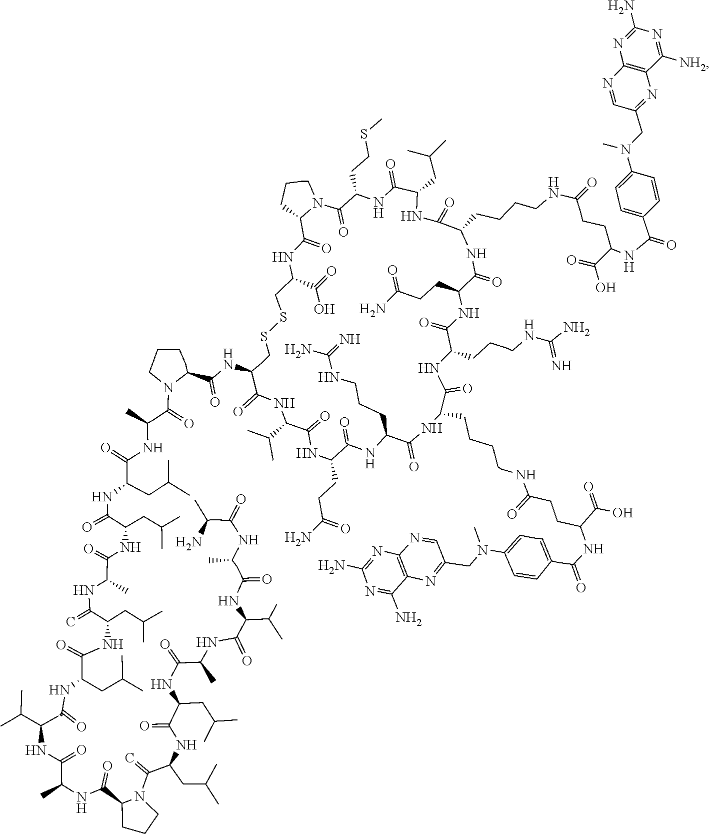 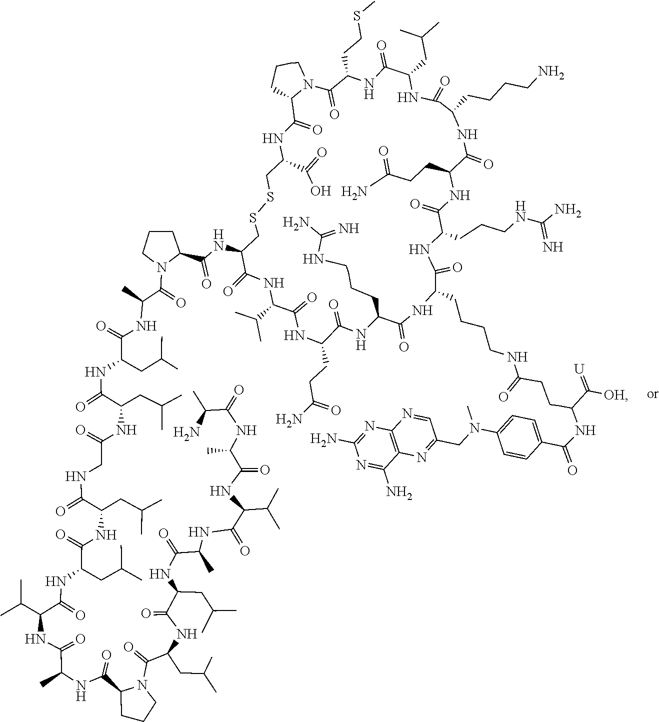 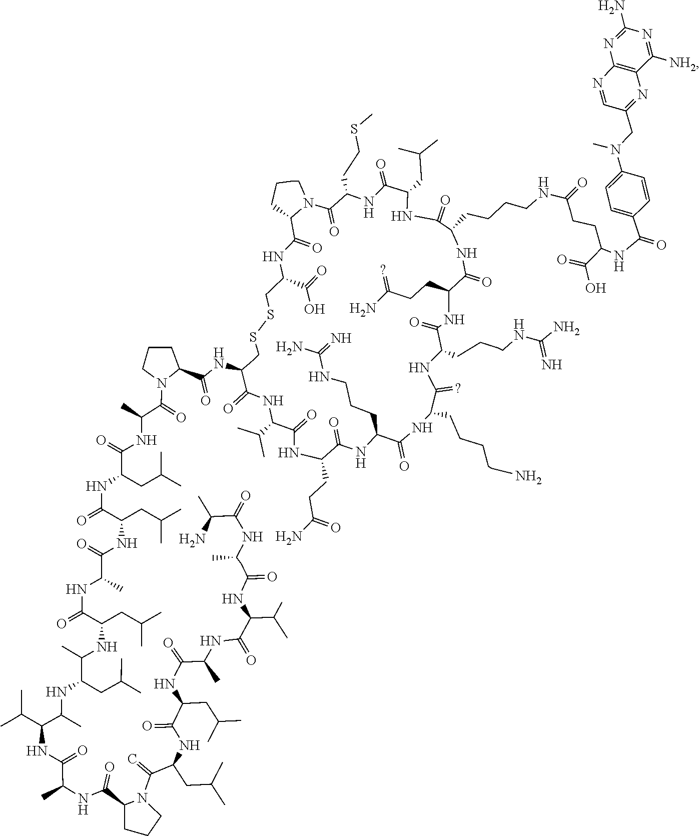
or a pharmaceutically acceptable salt thereof.

6. The compound of claim 1, having a formula: 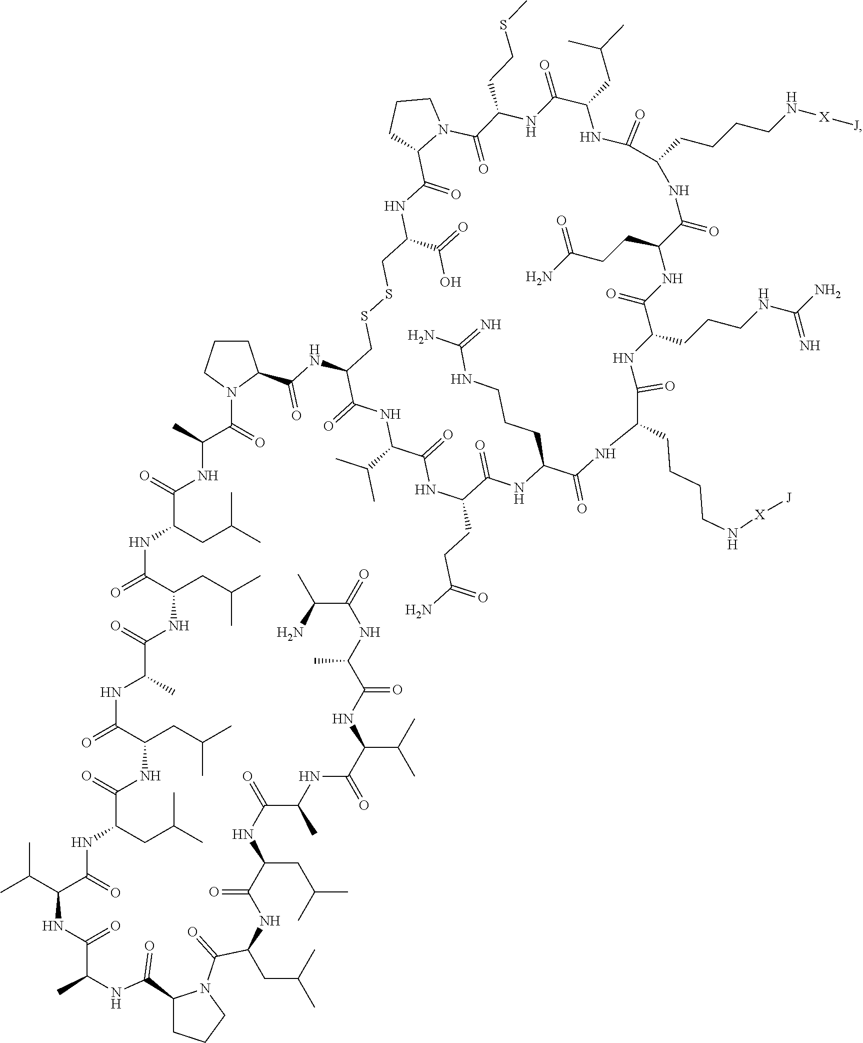 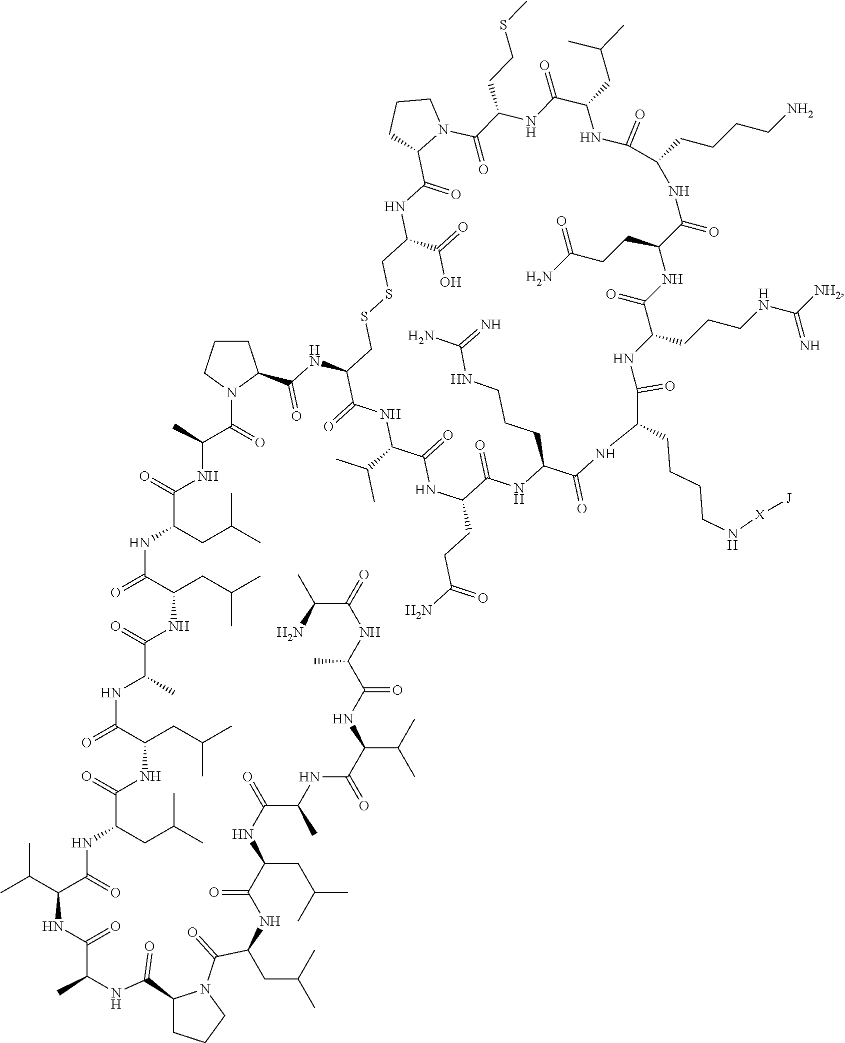 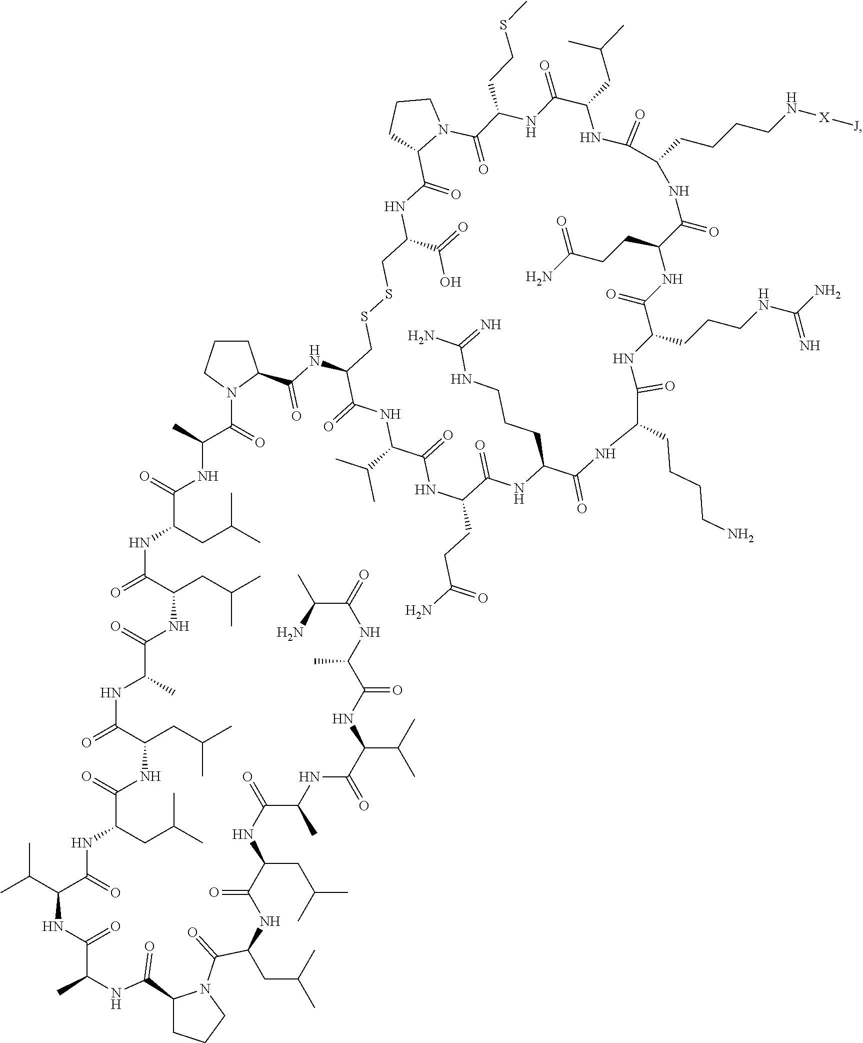 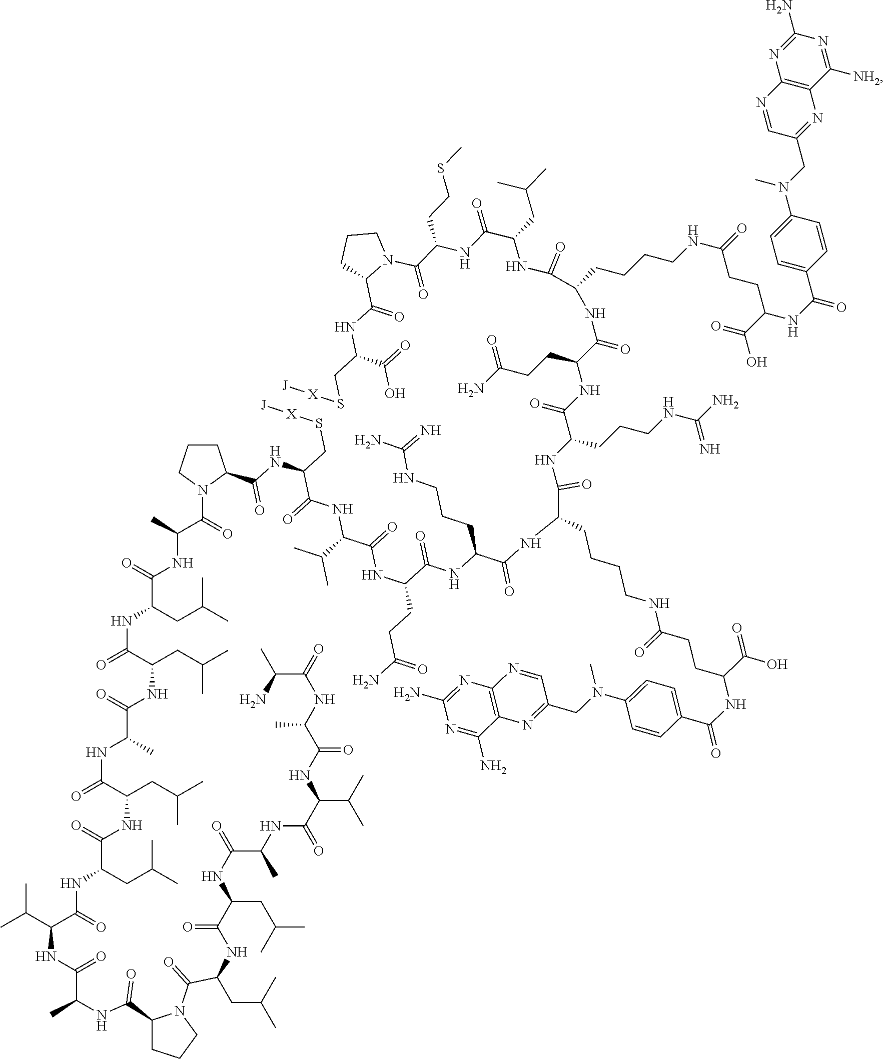 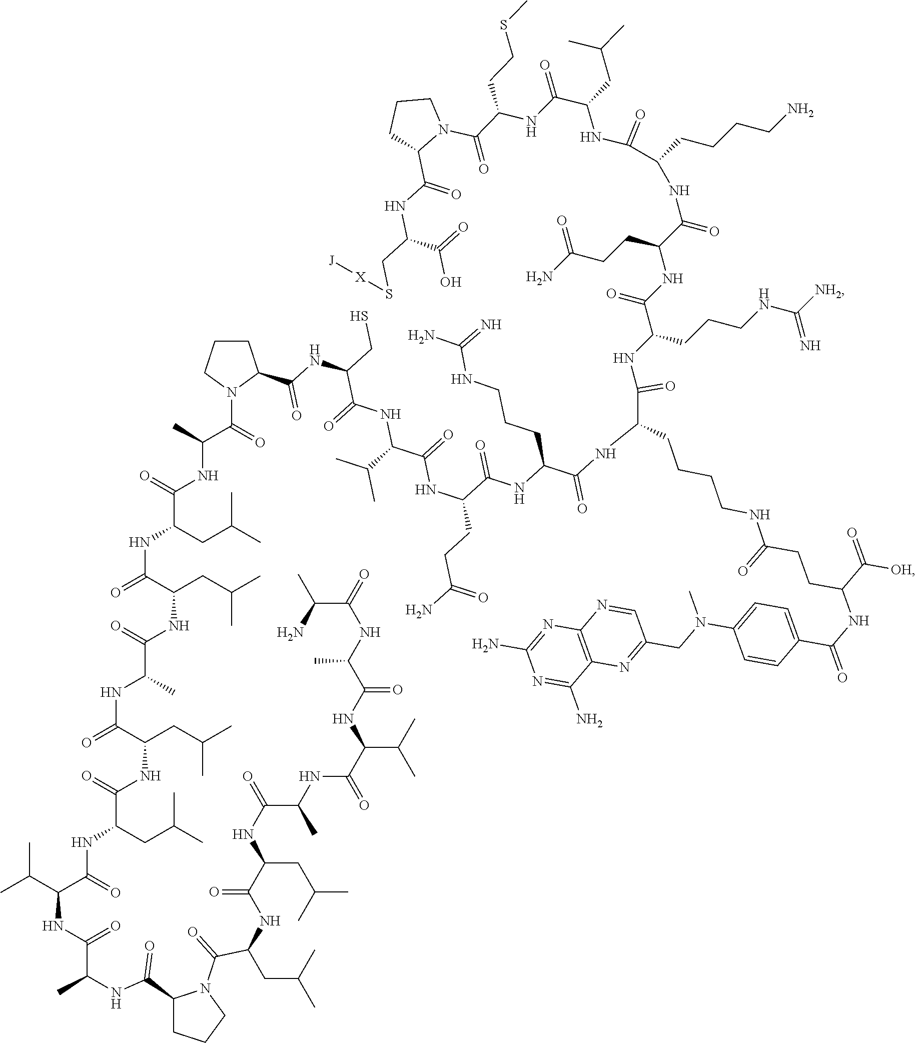 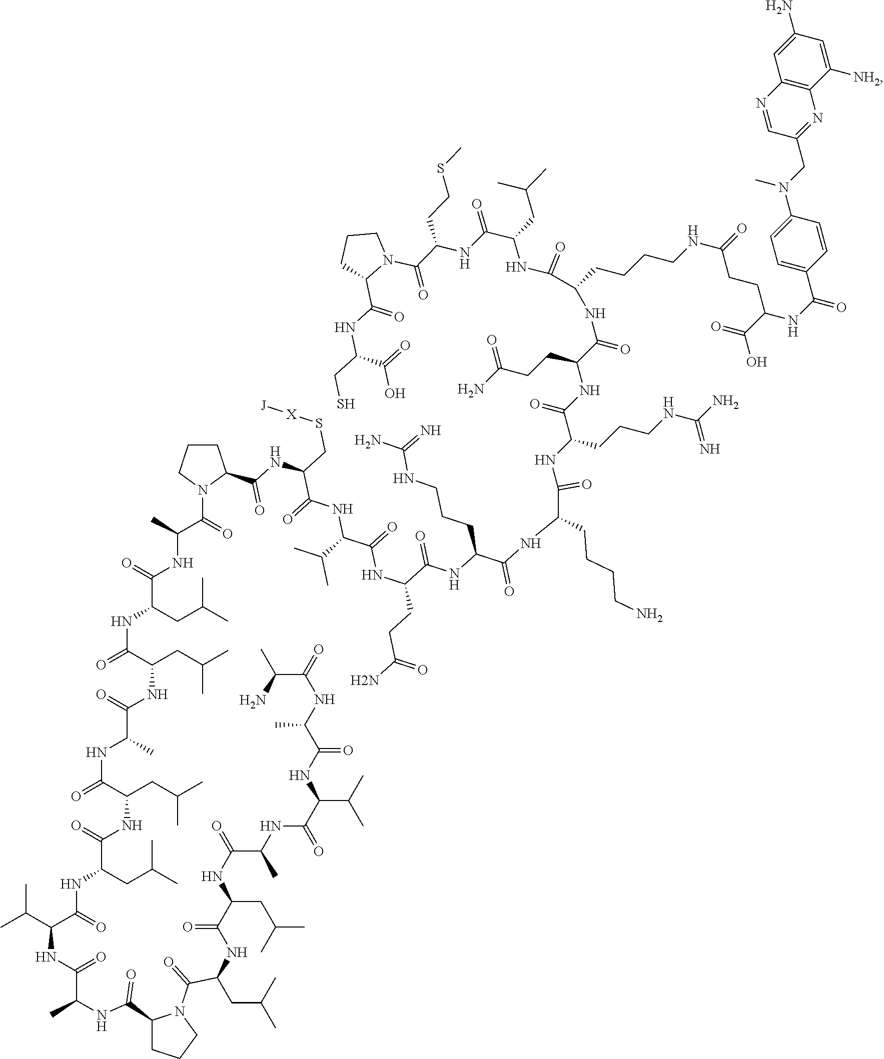 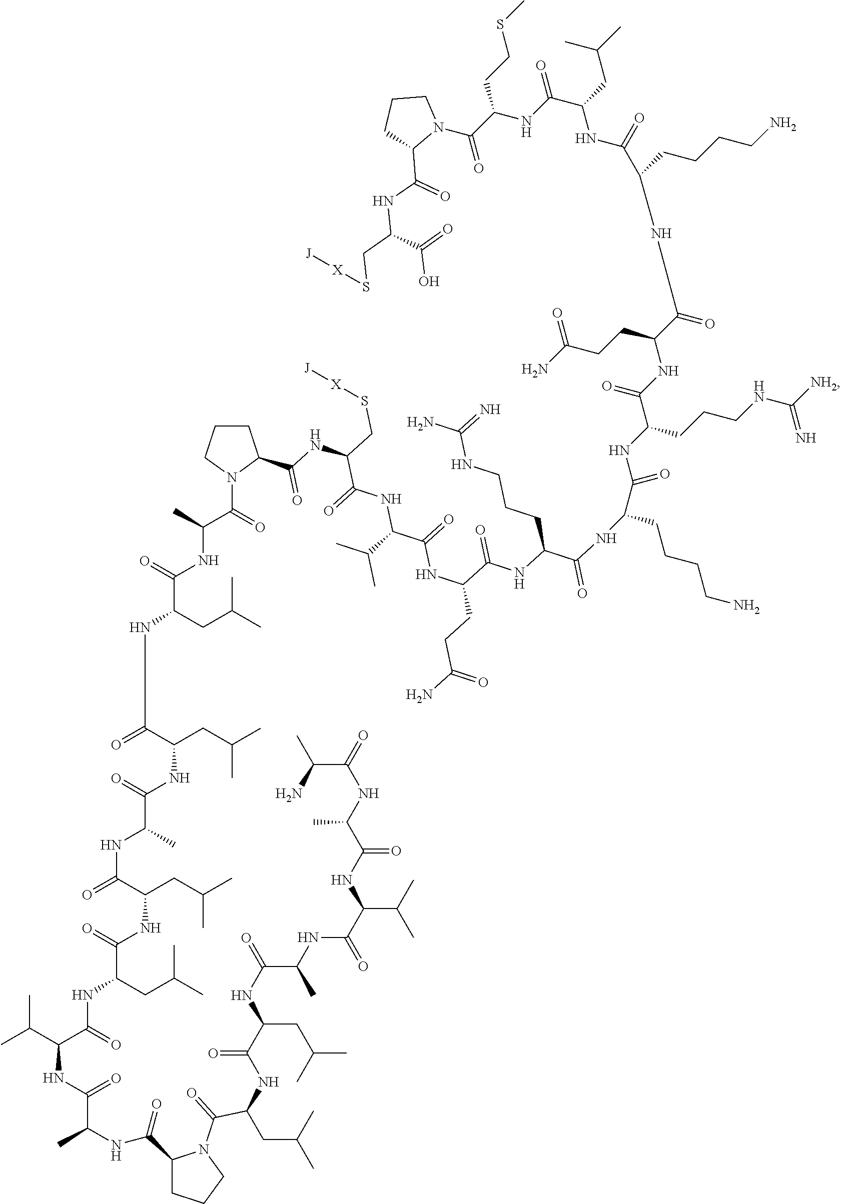 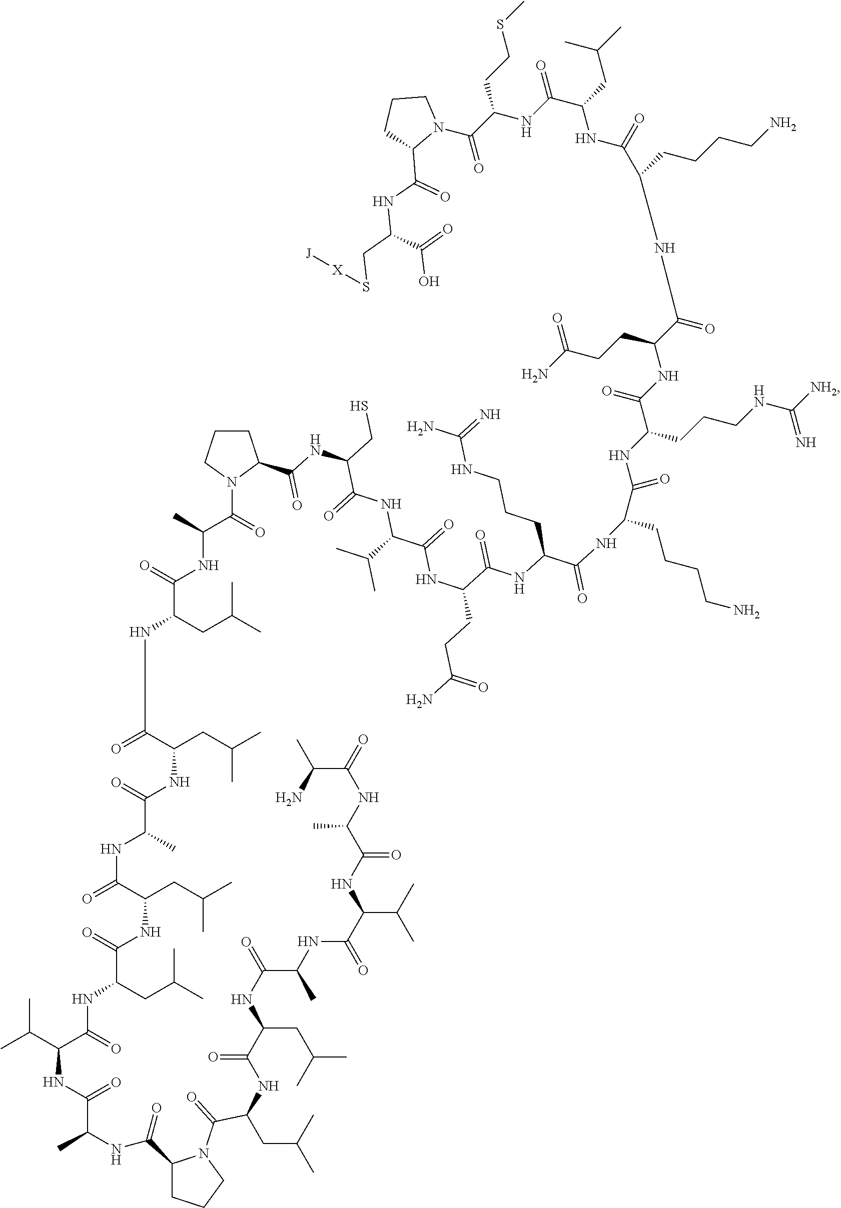 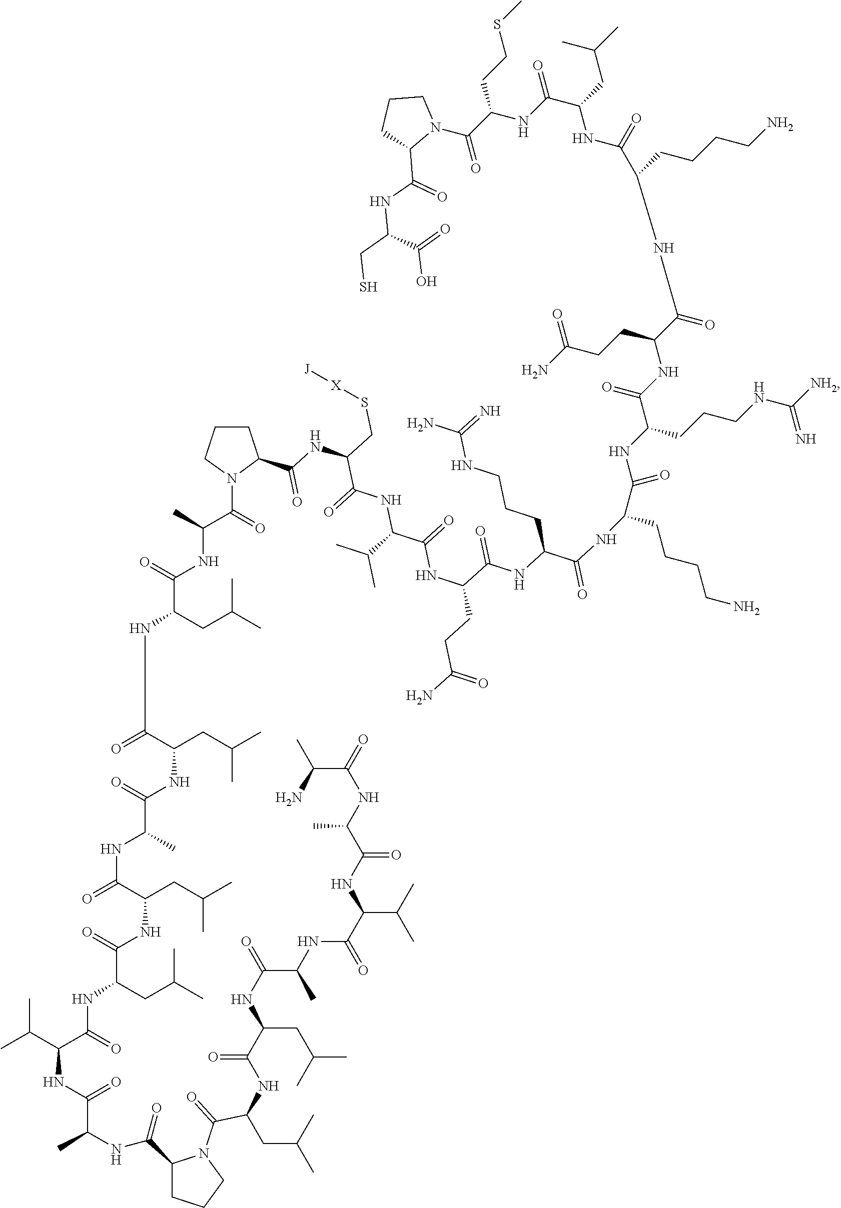 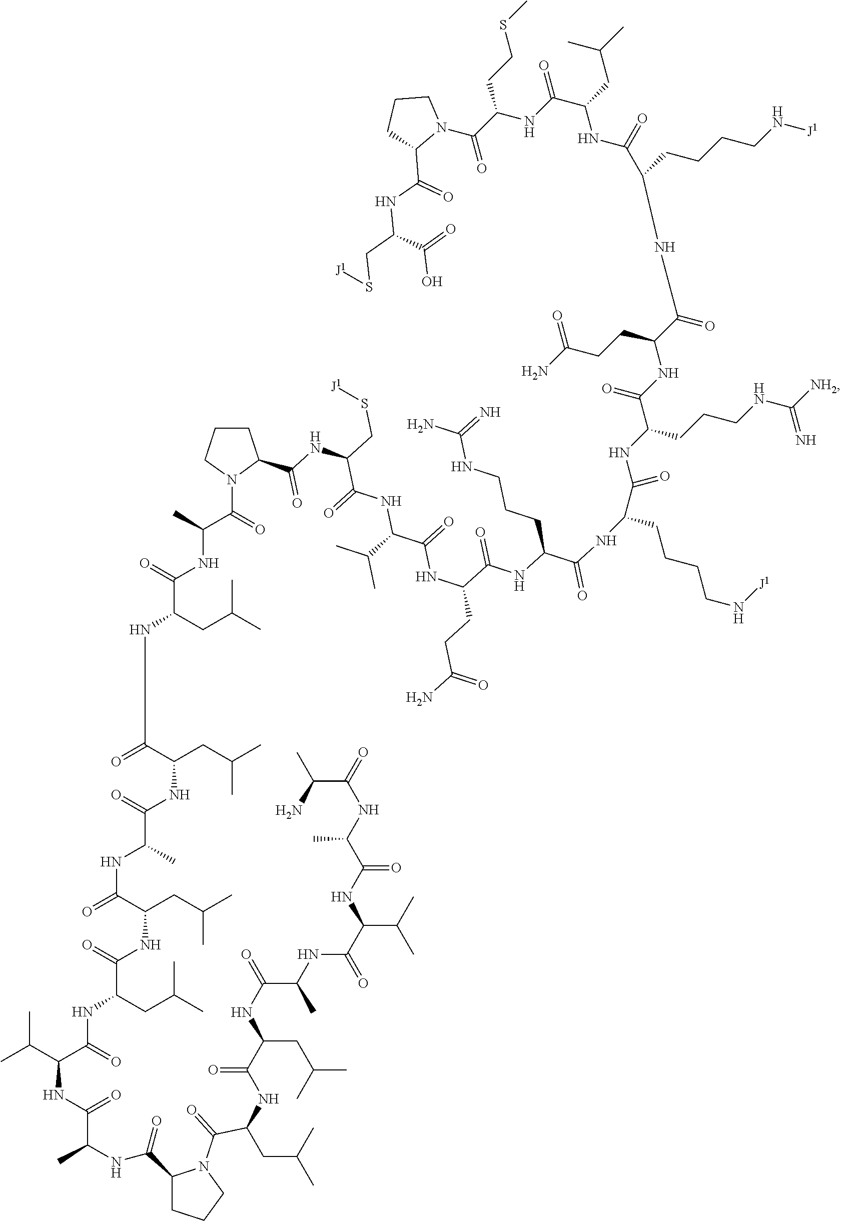 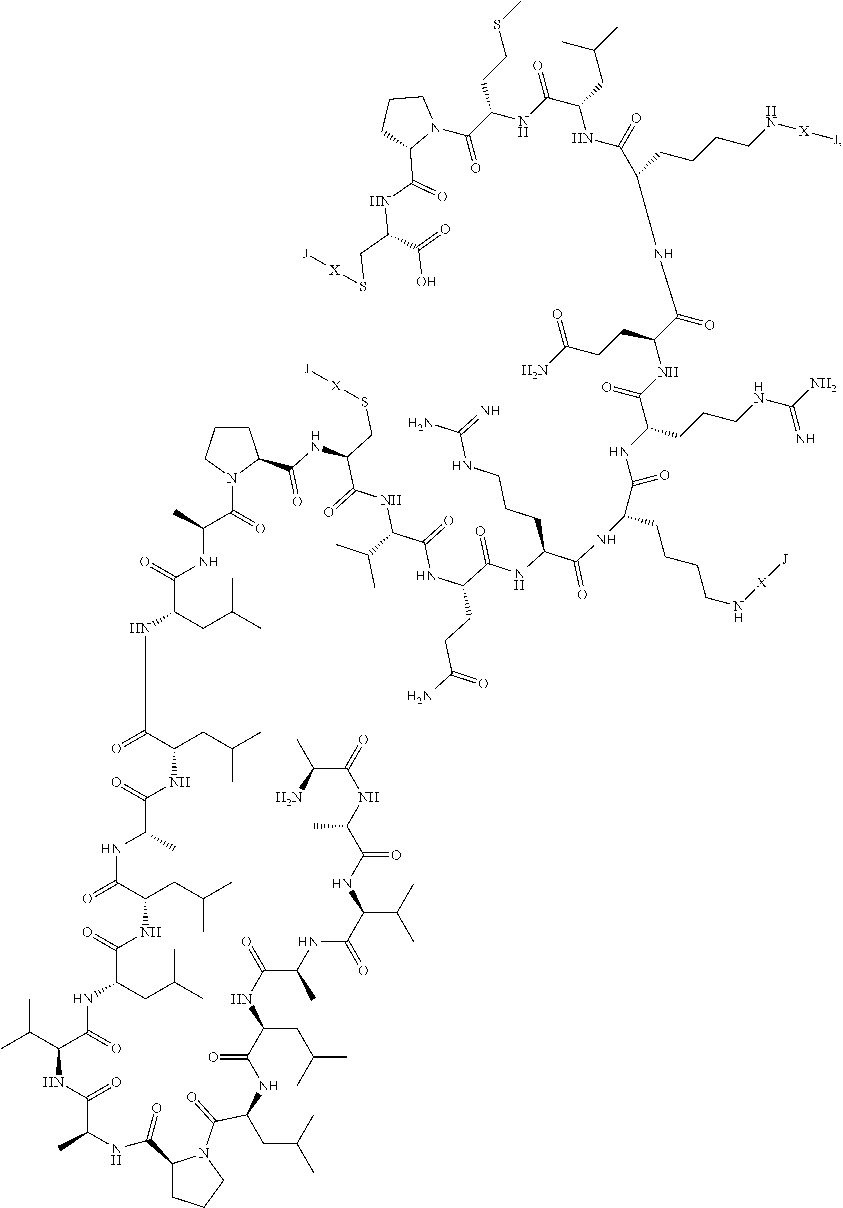 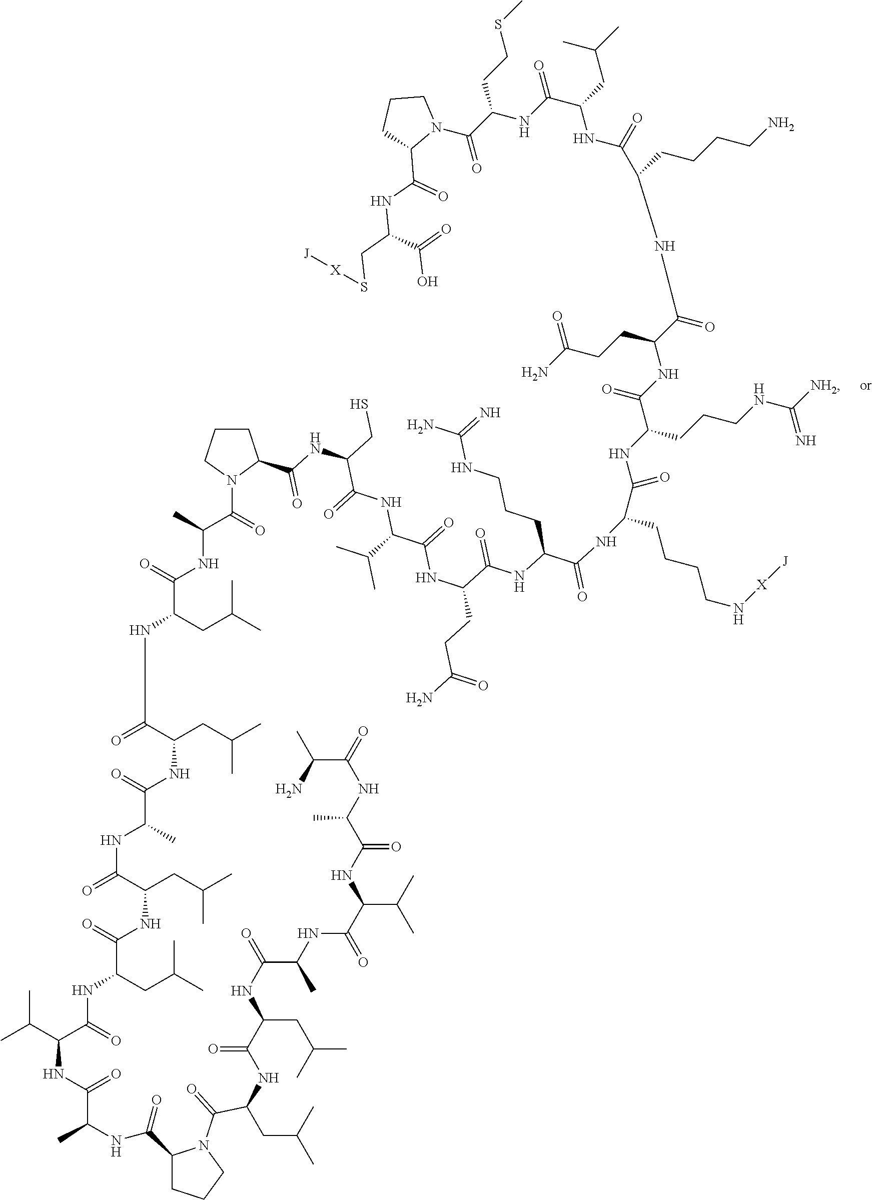 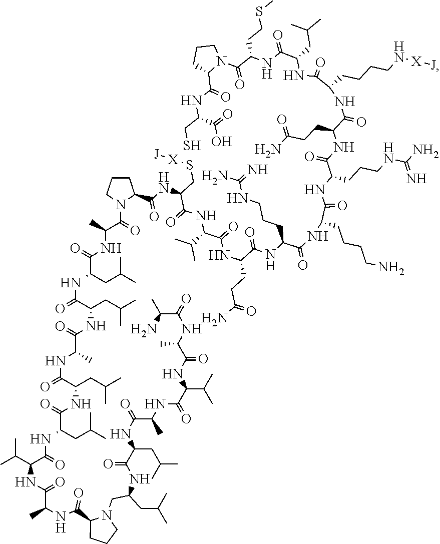
or a pharmaceutically acceptable salt thereof,
wherein
each J¹ is, independently, H or X-J, wherein one, two, or three of J¹ is H,
each X is, independently, a direct bond or a linker, and
each J comprises, independently, an active pharmaceutical agent.

7. The compound of claim 6, wherein J is a nuclease.

8. The compound of claim 7, wherein the nuclease is an RNAse.

9. The compound of claim 2, wherein Z comprises at least one lysine or at least one cysteine residue, and the side chain of the lysine is connected to X-J by an amide covalent connection or the side chain of the cysteine is connected to X-J by a disulfide covalent connection.

10. The compound of claim 1 or 9, wherein each X-J, independently, comprises an RNase, 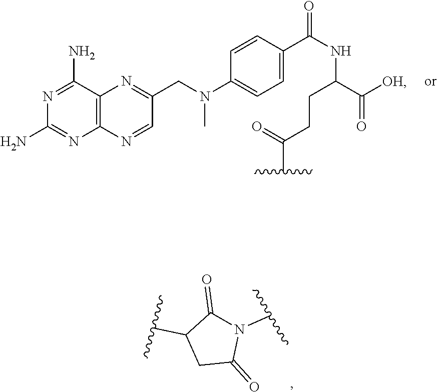 [SMILES: *C(=O)CCC(NC(=O)c1ccc(N(C)Cc2cnc3nc(N)nc(N)c3n2)cc1)C(=O)O ; *C1CC(=O)N(*)C1=O]
or a combination thereof.

11. A composition, comprising the compound of one of claims 1-10.

12. The composition of claim 11, which is a pharmaceutical composition further including a pharmaceutically acceptable carrier.

13. A method, comprising administering the compound of one of claims 1-10 or the composition of claim 11 or 12 to the subject.

14. A method of treating a disease in a subject in need thereof, comprising administering a therapeutically effective amount of the compound of one of claims 1-10 or the composition of claim 11 or 12 to the subject.

15. The method of claim 14, wherein the disease is cancer, psoriasis, rheumatoid arthritis, atopic dermatitis, an autoimmune disease, or an inflammatory disease.

16. The method of claim 15, wherein the cancer is a leukemia or a skin cancer, and optionally wherein the administration is topical administration and the compound is formulated as a cream formulation.

17. The method of claim 14, wherein the disease includes a leukemia, breast cancer, skin cancer, head and neck cancer, lung cancer, uterus cancer, psoriasis, rheumatoid arthritis, atopic dermatitis, an autoimmune disease, or an inflammatory disease.

18. The method of one of claims 13-17, wherein the administration is oral or intravenous administration.
